(19) United States
(12) Patent Application Publication
(10) Pub. No.: US 2008/0319089 A1
(43) Pub. Date: Dec. 25, 2008

(54) Method of colouring carrier materials

(76) Inventors: Martin Muller, Lorrach (DE); Didier Bauer, Kembs (FR)

Correspondence Address: JoAnn Villamizar;Ciba Corporation/Patent Department, 540 White Plains Road, P.O. Box 2005, Tarrytown, NY, 10591, US

(21) Appl. No.: 12/220,056

(22) Filed: Jul. 21, 2008

Related U.S. Application Data
(62) Division of application No. 10/529,803, filed on Mar. 30, 2005.

(30) Foreign Application Priority Data
Oct. 9, 2002 (EP) 02405868.7

(51) Int. Cl. A61K 8/19; C04B 14/04; C04B 14/34; A61Q 99/00; C09D 11/02; C04B 35/14

(52) U.S. Cl. 514/770; 106/482; 106/491; 106/404; 106/480; 106/403; 106/431; 106/31.85; 501/154

(57) ABSTRACT

The present invention relates to a method of producing coloured carrier particles which comprises which comprises:

a) dispersing a pigment in aqueous solution,
b) adding soda waterglass,
c) precipitating SiO₂ and the pigment onto the carrier particles by lowering the pH value.
Using the methods according to the invention it is possible to obtain colorations and coloured substrates that have surprisingly good light-fastness properties.

This is a divisional of U.S. application Ser. No. 10/529,803, pending, which is a 371 of PCT/EP03/10968 filed Oct. 2, 2003, which applications are hereby incorporated by reference.

The present invention relates to a method of producing coloured carrier materials (substrates), especially “effect pigments”, and to the coloured substrates obtained by such a method and also to the use thereof. Using the methods according to the invention, it is possible to obtain colorations and coloured substrates that have surprisingly good light-fastness properties.

Colour pigments and effect pigments are currently enjoying increasing popularity and are a decorative constituent of buildings or means of transport or are used for the optical enhancement of articles of practical use or artistic works. Also, besides the known typical effects, such as, for example, a metallic sheen in copper, bronze, silver or gold hues, pearlescent effects and interference effects, there is, especially, a growing interest in additionally imparting a brightly coloured appearance to such pigments.

Various approaches to satisfying that interest have been taken.

EP-A-810 270 describes a method of colouring aluminium, wherein a solution comprising an acid or an ammonium salt thereof is mixed with an aluminium pigment, whereupon the acid or its ammonium salt is adsorbed onto the surface of the aluminium pigment, and the aluminium pigment obtained is then added to a dispersion of a colour pigment in a non-polar solvent.

In accordance with DE-A-4 225 357, organic pigments and dyes which are soluble in acids without undergoing decomposition are, by means of neutralisation of a dispersion thereof which also comprises substrate particles, precipitated onto the substrate particles.

DE-A-4 225 031 describes a method of producing coloured gloss pigments, wherein substrate particles are dispersed in a solution of the pigment and the pigment is precipitated directly onto the surface of the substrate particles by adding a solvent in which the pigment is insoluble. In the examples, the solvents used are, for the most part, strong acids such as sulfuric acid, polyphosphoric acid or trifluoroacetic acid.

It is furthermore known from DE-A-4 009 567 to deposit colorants such as phthalocyanine and metal phthalocyanine pigments onto a silicate substrate from concentrated sulfuric acid by adding water. Finally, it is also known from DE-A-3 536 168 for vat dyes, in particular 4-methyl-6-chloro-6′-methoxythioindigo, present in the leuco form of the dye in an alkaline solution, to be precipitated from that solution onto the substrate whilst being oxidised to the coloured form of the dye.

U.S. Pat. No. 5,718,753 describes coloured metallic pigments, for example aluminium flakes, that are encapsulated in a continuous homogeneous layer of organic pigment particles. The metallic pigments are produced by means of PVD (Physical Vapour Deposition) of the organic pigments onto the aluminium flakes.

U.S. Pat. No. 5,156,678 describes the colouring of effect pigments with metal-free phthalocyanine dyes or with phthalocyanine dyes. The dye is dissolved in sulfuric acid and deposited onto the effect pigment by adding water.

The use of adhesion promoters such as resin binder systems (EP-A403 432), anionic polymers and chromium salts (U.S. Pat. No. 4,755,229, U.S. Pat. No. 5,931,996) or aluminium hydroxide (DE-A-2 429 762) usually results in dulling of the pigment.

In those methods, the low pigment-substrate affinity, the weak pigment character of the precipitated products and the lack of universal applicability are disadvantageous.

It has now been found, surprisingly, that the adhesion to various substrates and the light-fastness properties can be greatly increased when the substrates are coloured as described hereinbelow.

The present invention accordingly relates to a method of producing coloured carrier materials (carrier particles), which comprises

a) dispersing the carrier particles in a solution of a colorant or latent pigment, adding the carrier particles to a solution of a colorant or latent pigment or adding a latent pigment or a colorant to a dispersion of the carrier particles,
b) precipitating the colorant or latent pigment onto the carrier particles, and
c) in the case of a latent pigment, subsequently converting it to the pigment;
to coloured carrier particles obtainable by such a method, and also to the use of the coloured carrier particles in the colouring of coating compositions, printing inks, plastics, glass, ceramic products and cosmetic preparations.

Advantageously, the method comprises

a) adding the carrier particles to the solution of the colorant or latent pigment,
b) precipitating the colorant or latent pigment onto the carrier particles and
c) in the case of a latent pigment, subsequently converting it to the pigment;
and also coloured carrier particles obtainable by such a method.

The method according to the invention is in principle suitable for colouring any carrier material (substrate). The sole requirement is that the carrier particles should be stable in the solvent without undergoing decomposition. Examples of carrier particles are cellulose (wood), keratin (hair) and anodized aluminium.

The carrier particles are preferably selected from metallic, metal oxide, non-metallic, and (non-metal) oxide carrier particles, especially effect pigments, polymeric compounds and combinations thereof and organic or inorganic pigments.

In general 15 to 100 parts by weight of colorant are used per 100 parts by weight of carrier particles. If the aluminium flakes or flakes on the basis of SiO₂ substrates, such as Iriodin® pigments are used as carrier particles, in particular 20 to 80 parts by weight of colorant are used per 100 parts by weight of carrier particles.

Preference is given to flake-like substrates, the particles of which generally have a length of from 2 μm to 5 mm, a width of from 2 μm to 2 mm and a thickness of from 20 nm to 1.5 μm and a ratio of length to thickness of at least 2:1. It is presently preferred that the diameter of the flakes be in a preferred range of about 2 to 60 μm with a more preferred range of about 5-40 μm.

Metallic effect pigments (metal effect pigments) generally are flake-like particles of aluminium, copper, zinc, gold bronze, titanium (EP-A-796 688), zirconium, tin, iron (EP-A-673 980) or steel or pigments of alloys of the afore-mentioned metals. However, particle shapes other than those of metal flakes are known, such as those of spherical zinc dust or dendritic copper powder. Preference is given to metal flakes, such as aluminium flakes, copper/tin flakes, copper flakes and copper/zinc flakes. Special preference is given to aluminium flakes and coloured aluminium flakes, such as Silberline® (Silberline Inc.), Aloxal 3010 and Metallux 2154 (Carl Eckart GmbH & Co.).

Examples of metal oxide effect pigments are effect pigments based on aluminium oxide (Al₂O₃), zinc oxide (ZnO), zirconium oxide (ZrO₂), titanium dioxide (TiO₂), indium oxide (In₂O₃), indium tin oxide (ITO), tantalum pentoxide (Ta₂O₅), cerium oxide (CeO₂), yttrium oxide (Y₂O₃), europium oxide (Eu₂O₃), iron oxides, such as Fe₃O₄ and Fe₂O₃, hafnium oxide (HfO₂), lanthanum oxide (La₂O₃), magnesium oxide (MgO), neodymium oxide (Nd₂O₃), praseodymium oxide (Pr₆O₁₁), samarium oxide (Sm₂O₃), antimony trioxide (Sb₂O₃), tin oxide (SnO₂), tungsten trioxide (WO₃) or combinations thereof, or effect pigments comprising layers of the afore-mentioned metal oxides.

Examples of (non-metal) oxide effect pigments are effect pigments based on SiOₓ wherein 0.03≦x≦0.95 or SiO_{y} wherein 0.95<y≦2.0, especially silicon monoxide (SiO), silicon dioxide (SiO₂) or selenium trioxide (Se₂O₃), or effect pigments comprising layers of the afore-mentioned materials such as, for example, SiO₂ flakes.

Examples of non-metallic effect pigments are effect pigments based on BC, BN, SiC or Si₃N₄, or effect pigments comprising layers of the afore-mentioned materials (see, for example, EP 02 405 649.1).

Further examples are effect pigments based on metal fluorides, such as magnesium fluoride (MgF₂), aluminium fluoride (AlF₃), cerium fluoride (CeF₃), lanthanum fluoride (LaF₃), zinc sulfide (ZnS), carbon, hafnium nitride (HfN), hafnium carbide (HfC), sodium aluminium fluorides (for example, Na₃AlF₆ or Na₅Al₃F₁₄), neodymium fluoride (NdF₃), samarium fluoride (SmF₃), barium fluoride (BaF₂), calcium fluoride (CaF₂), lithium fluoride (LiF) or combinations thereof, and effect pigments comprising layers of the afore-mentioned materials, and effect pigments comprising organic monomers and polymers, including dienes or alkenes, such as acrylates (for example, methacrylates), perfluoroalkanes, polytetrafluoroethylene (Teflon), fluorinated ethylenepropylene (FEP) and combinations thereof.

In the case of multi-layer pigments, the afore-mentioned materials, especially metals, metal oxides and (non-metal) oxides, may be included in the layered structure, wherein layers comprising Al, SiOₓ (0.03≦x≦0.95), SiO_{y} (0.95<y≦2.0), TiO₂ and SiO₂ (see, for example, EP-A-803 549, PCT/EP03/02196, PCT/EP03/09296 and WO03/68868) are preferred and colour-imparting oxides of metals such as Fe, Co, Mn, Sn, Cr, Ni, Cu are also possible.

Examples of preferred effect pigments are pearlescent pigments based on the natural mineral mica or on mica already encapsulated in a thin layer of metal oxide, for example titanium dioxide and/or iron oxide, such as Iriodin® 9103 (Merck), Xirallic®) effect pigments (Merck) based on aluminium oxide (Al₂O₃) flakes or on Al₂O₃ flakes already coated with metal oxides, and Colorstream® pigments based on synthetically produced SiO₂ (silicon dioxide) flakes already covered with metal oxides.

The method according to the invention comprises, on the one hand, the colouring of substrates using colorants (method A) and, on the other hand, the colouring of substrates using latent pigments (method B), which are described in detail hereinbelow.

### Method (A)

Method (A) comprises

a) dispersing the carrier particles in a solution of a colorant, adding the carrier particles to a solution of a colorant or adding a colorant to a dispersion of the carrier particles,
b) precipitating the colorant onto the carrier particles.

In a preferred embodiment, the present invention relates to a method of producing coloured carrier particles, which comprises

a1) dissolving a colorant in a solvent,
a2) adding the carrier particles to the solution prepared in Step a1),
b) precipitating the colorant onto the carrier particles.

In method (A), either there is used a colorant which is soluble in an alkaline medium and which, in Step b), is precipitated onto the substrate by adding acid and/or metal salts, or there is used a colorant which is soluble in a weakly acid or neutral medium and which, in Step b), is precipitated onto the substrate by adding acid and/or metal salts.

The method according to the invention makes use of the pH-dependent solubility or differing solubility of the salts of the colorants, there being achieved a high affinity with the substrate and, where applicable, π-π stabilisation of the deposited colorants by means of suitable functional groups and modifications.

In general, a colorant is used which is soluble in an alkaline medium and which, in Step b), is precipitated onto the substrate by adding acid and/or ammonium or metal salts, such as an alkali metal salt, alkaline earth metal salt, aluminium salt or transition metal salt, especially NH₄⁺, ⁺NR³¹R³²R³³R³⁴, wherein R³¹, R³², R³³ and R³⁴ are as defined hereinbelow, Na, K, Mg, Ca, Sr, Ba, Mn, Cu, Ni, Cd, Co, Cr, Zn, Al or Fe, or a colorant is used which is soluble in a weakly acidic or neutral medium and which, in Step b), is precipitated onto the substrate by adding acid and/or metal salts, such as an alkali metal salt, alkaline earth metal salt, aluminium salt or transition metal salt, especially NH₄⁺, ⁺NR³¹R³²R³³R³⁴, wherein R³¹, R³², R³³ and R³⁴ are as defined hereinbelow, Na, K, Mg, Ca, Sr, Ba, Mn, Cu, Ni, Cd, Co, Cr, Zn, Al or Fe. Special preference is given to salts of calcium and aluminium.

Examples of such colorants are described, for example, in PCT/EP02/04071 and PCT/EP03/00817.

The colorant is generally selected from compounds of the following formula

D(SO₂NHE)_{y}(II) or salts of the formula D(SO₂NE Cat)_{y} (II′),

wherein
y is an integer from 1 to 8,
D is the radical of a chromophore of the 1-aminoanthraquinone, anthraquinone, anthrapyrimidine, azo, azomethine, benzodifuranone, quinacridone, quinacridone quinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone or thioindigo series,
Cat is an alkali metal cation or an ammonium cation, and
E is any desired group suitable for imparting solubility in an alkaline medium,
and from compounds of the following formula

D(F)_{y}(III),

wherein
y is an integer from 1 to 8,
D is the radical of a chromophore of the 1-aminoanthraquinone, anthraquinone, anthrapyrimidine, azo, azomethine, benzodifuranone, quinacridone, quinacridone quinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone or thioindigo series, and
F is any desired group suitable for imparting solubility in an aqueous medium, such as, for example, —SO₃M or —COOM, wherein M is a cation or hydrogen.

Preference is given to colorants of formula II and II′ over those of formula III.

E is generally a hydrogen atom, a branched or straight-chain C₁₋₈alkyl, C₂₋₈alkenyl, C₂₋₈alkynyl, aryl, nitrogen-, oxygen- or sulfur-containing 5- or 6-membered heterocyclic ring, C₁₋₈-alkyl-arylene, aryl-C₁₋₈alkylene or aryl-L-arylene radical, which may be substituted by one or more —OH, —OCat, —SH, —SCat, —OR¹, —SR², —C(O)OR³, —C(O)OCat, —NO₂, —C(O)R⁴ and/or —NR⁵R⁶ groups, the C₁₋₈-alkyl radical being uninterrupted or interrupted one or more times by —O— or —S—, and wherein R¹, R², R³, R⁴ are each independently of the others a C₁₋₈-alkyl radical, C₇₋₁₂aralkyl radical or C₆₋₁₂aryl radical and, in addition, R³ and R⁴ may be a hydrogen atom, and R⁵ and R⁶ are each independently of the other a hydrogen atom, a C₁₋₈alkyl radical, a C₁₋₄alkoxy-C₁₋₄alkyl radical, a C₆₋₁₂aryl radical, a C₇₋₁₂aralkyl radical or a radical —(CH₂)ₒH wherein o is an integer from 2 to 6.

In the groups E, the radicals may have the following meanings:

Alkyl or alkylene may be straight-chain or branched.

C₁₋₈-Alkyl is, for example, methyl, ethyl, n-propyl, isopropyl, n-butyl, sec-butyl, isobutyl, tert-butyl, n-pentyl, 2-pentyl, 3-pentyl, 2,2-dimethylpropyl, hexyl, heptyl, 2,4,4-trimethylpentyl, 2-ethylhexyl or octyl, which are unsubstituted or substituted by —OH, —OCat, —SH, —SCat, —OR¹, —SR², —C(O)OR³, —C(O)R⁴ or —NR⁵R⁶, wherein R¹, R², R³, R⁴, R⁵ and R⁶ are as defined hereinbefore.

Examples of C₂₋₈alkenyl, which may also have two double bonds in an isolated or conjugated arrangement, as appropriate, are vinyl, allyl, 2-propen-2-yl, 2-buten-1-yl, 3-buten-1-yl, 1,3-butadien-2-yl, 2-penten-1-yl, 3-penten-2-yl, 2-methyl-1-buten-3-yl, 2-methyl-3-buten-2-yl, 3-methyl-2-buten-1-yl or 1,4-pentadien-3-yl, which are unsubstituted or substituted by —OH, —OCat, —SH, —SCat, —OR¹, —SR², —C(O)OR³, —C(O)R⁴ or —NR⁵R⁶, wherein R¹, R², R³, R⁴, R⁵ and R⁶ are as defined hereinbefore.

Special preference is given to linear C₁₋₅alkyl and C₂₋₅alkenyl radicals terminally substituted by a group —OH, —OCat, —SH, —SCat, —OR¹, —SR², —C(O)OR³, —C(O)R⁴ or —NR⁵R⁶.

C₂-C₈Alkyl which is interrupted one or more times by —O— or —S— is interrupted, for example, 1, 2 or 3 times by —O— and/or —S—, resulting in, for example, structural units such as —(CH₂)₂OCH₃, —(CH₂CH₂O)₂CH₂CH₃, —CH₂—O—CH₃, —CH₂CH₂—O—CH₂CH₃, —[CH₂CH₂O]_{y}—CH₃ wherein y is from 1 to 3, —CH₂—CH(CH₃)—O—CH₂—CH₂CH₃ or —CH₂—CH(CH₃)—O—CH₂—CH₃, which are unsubstituted or substituted by —OH, —OCat, —SH, —SCat, —OR¹, —SR², —C(O)OR³, —C(O)R⁴ or —NR⁵R⁶.

Examples of C₂₋₈alkynyl are ethynyl, 1-propyn-1-yl, 2-butyn-1-yl, 3-butyn-1-yl, 2-pentyn-1-yl and 3-pentyn-2-yl.

C₁-C₈Alkylene is linear or branched alkylene, such as, for example, methylene, ethylene, propylene, isopropylene, n-butylene, sec-butylene, isobutylene, tert-butylene, pentylene, hexylene, heptylene, —CH(CH₃)—CH₂—, —CH(CH₃)—(CH₂)₂—, —CH(CH₃)—(CH₂)₃—, —C(CH₃)₂—CH₂— and

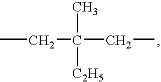

alkylene radicals having from 1 to 5 carbon atoms being preferred.

Alkylene radicals interrupted by —O— or —S— result in, for example, structural units such as —CH₂—O—CH₂—, —CH₂CH₂—O—CH₂CH₂—, —CH₂—CH(CH₃)—O—CH₂—CH(CH₃)—, —CH₂—S—CH₂—, —CH₂CH₂—S—CH₂CH₂— and —CH₂CH₂CH₂—S—CH₂CH₂CH₂—.

Examples of a C₁₋₈alkoxy radical which may be linear or branched are methoxy, ethoxy, n-propoxy, isopropoxy, n-butoxy, sec-butoxy, isobutoxy, tert-butoxy, n-pentyloxy, 2-pentyloxy, 3-pentyloxy, 2,2-dimethylpropoxy, n-hexyloxy, n-heptyloxy, n-octyloxy, 1,1,3,3-tetramethyl-butoxy and 2-ethylhexyloxy.

According to the present invention, aryl is understood to be especially an aryl radical containing from 6 to 14 carbon atoms, examples being phenyl, naphthyl and biphenyl, which are unsubstituted or substituted one, two or three times by linear or branched C₁₋₄alkyl, such as methyl, ethyl, n-propyl, isopropyl, n-butyl, isobutyl, sec-butyl and tert-butyl, by linear or branched C₁₋₄alkoxy, such as methoxy, ethoxy, n-propoxy, isopropoxy, n-butoxy, isobutoxy, sec-butoxy and tert-butoxy, by linear or branched C₁₋₄alkylthio, such as methylthio, ethylthio, n-propylthio, isopropylthio, n-butylthio, isobutylthio, sec-butylthio and tert-butylthio, —NO₂, —CO₂H, —COOCat, —OH, —SH, —OCat, —SCat or a group (CH₂)e-E¹, wherein e is an integer from 1 to 6, especially 2 or 3, and E¹ is a hydrogen atom, a group —OH, —OCat, —SH, —SCat, —OR¹, —SR², —C(O)OR³, —C(O)R⁴ or —NR⁵R⁶, wherein R¹, R², R³ and R⁴ are each independently of the others a C₁₋₄alkyl radical, especially methyl or ethyl, and R⁵ and R⁶ are a radical —(CH₂)ₒOH, wherein o is an integer from 2 to 6, especially 2 or 3, and Cat is an alkali metal cation, especially a sodium or potassium cation, unsubstituted ammonium or an ammonium cation.

Preference is given to phenyl groups which can be substituted by one, two or three groups selected from —NO₂, —CO₂H, —COOCat, —OH, methoxy, —(CH₂)₂OH, —OCat and —(CH₂)₂Ocat. Examples of a C₇₋₁₂aralkyl radical which is unsubstituted or substituted are benzyl, 2-benzyl-2-propyl, β-phenyl-ethyl, α,α-dimethylbenzyl and ω-phenyl-butyl.

An oxygen-, sulfur- or nitrogen-containing, 5- or 6-membered heterocyclic ring is, for example, pyrrolyl, oxinyl, dioxinyl, 2-thienyl, 2-furyl, 1-pyrazolyl, 2-pyridyl, 2-thiazolyl, 2-oxazolyl, 2-imidazolyl, isothiazolyl, triazolyl or any other ring system which consists of thiophene, furan, pyridine, thiazole, oxazole, imidazole, isothiazole, thiadiazole, triazole, pyridine and benzene rings and is unsubstituted or substituted by from 1 to 6 ethyl, methyl, ethylene and/or methylene substituents.

SO₂NHE, SO₂NECat and F within a colorant of formula II, II′ and III may have different substituent meanings.

Suitable cations Cat are, generally, radicals which are capable of forming water-soluble salts with sulfonamides, for example alkaline earth metal cations, such as strontium and calcium cations, alkali metal cations, especially lithium, sodium and potassium cations, and quaternary ammonium cations, especially substituted ammonium and ammonium cations of the formula ⁺NR³¹R³²R³³R³⁴ wherein R³¹, R³², R³³ and R³⁴ are each independently of the others a hydrogen atom, a straight-chain or branched C₁₋₁₆alkyl radical which is unsubstituted or substituted by one or more C₁₋₄alkoxy radicals, a straight-chain or branched C₂₋₁₆alkenyl radical, a hydroxy-C₁₋₈alkyl radical, especially a hydroxy-C₁₋₄alkyl radical, or a C₆₋₁₂aryl radical which is unsubstituted or substituted by one or more C₁₋₄alkyl radicals, C₁₋₄alkoxy radicals or hydroxy groups, especially a phenyl group substituted by a hydroxy group, or a C₇₋₁₂aralkyl radical, such as phenyl-C₁₋₄alkyl, wherein at least one of the radicals R³¹, R³², R³³ and R³⁴ is other than a hydrogen atom, or two of the radicals R³¹, R³², R³³ and R³⁴ together with the nitrogen atom to which they are bonded form a 5- or 6-membered ring which may contain additional hetero atoms, such as, for example, sulfur, nitrogen and oxygen.

Examples of especially preferred ammonium cations are
unsubstituted ammonium,
mono-, di-, tri- or tetra-C₁₋₄alkylammonium, such as methylammonium, ethylammonium, 3-propylammonium, isopropylammonium, butylammonium, sec-butylammonium, isobutyl-ammonium, 1,2-dimethylpropylammonium and 2-ethylhexylammonium, dimethylammonium, diethylammonium, dipropylammonium, diisopropylammonium, dibutylammonium, diisobutyl-ammonium, di-sec-butylammonium, di-2-ethylhexylammonium, N-methyl-n-butylammonium and N-ethyl-n-butylammonium, trimethyl- and triethyl-ammonium, tripropylammonium, tributyl-ammonium, N,N-dimethylethylammonium, N,N-dimethylisopropylammonium, N,N-dimethyl-benzylammonium and (CH₃)₂((CH₃O)₂CHCH₂)NH⁺,
mono-, di-, tri- and tetra-C₈₋₁₆alkylammonium, such as

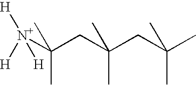

(idealized representation of the ammonium cation of Primene 81R®),
C₁₋₄alkoxy-C₁₋₄alkylammonium, such as 2-methoxyethylammonium, bis(2-methoxyethyl)-ammonium, 3-methoxypropylammonium and ethoxypropylammonium,
mono-, di- or tri-(hydroxy-C₁₋₄alkyl)ammonium, such as mono-, di- or tri-ethanolammonium, mono-, di- or tri-isopropanolammonium, N-methyl- or N,N-dimethyl-ethanolammonium, -propanolammonium or -isopropanolammonium, N-methyl-diethanolammonium, -dipropanolammonium or -diisopropylammonium, N-ethyl-diethanolammonium, -dipropanolammonium or -diisopropylammonium, and N-propyl-diethanolammonium, -dipropanolammonium or -diisopropylammonium,
N-(2-hydroxyethyl)pyrrolidinium, N-(2- or 3-hydroxypropyl)pyrrolidinium, N-(2-hydroxy-ethyl)piperidinium, N-(2- or 3-hydroxypropyl)piperidinium, N-(2-hydroxyethyl)morpholinium, N-(2- or 3-hydroxypropyl)morpholinium and N-(2-hydroxyethyl)piperazinium, and

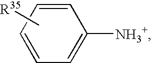

especially

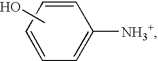

such as 2-, 3- or 4-hydroxyphenyl-ammonium, wherein R³⁵ is a hydroxy group, a C₁₋₈alkoxy group, a carboxylic acid group or —COOR³⁶, wherein R³⁶ is a C₁₋₈alkyl group, C₆₋₁₂aryl group or C₇₋₁₂aralkyl group. Ammonium cations of the formula

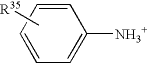

can contribute to an increase in light-fastness. Also suitable are polyammonium salts, especially diammonium compounds. Preferred diammonium compounds are derived from the following amines: 1,2-diaminoethane, 1,2-diamino-1-methylethane, 1,2-diamino-1,2-dimethylethane, 1,2-diamino-1,1-dimethylethane, 1,2-diaminopropane, 1,3-diaminopropane, 1,3-diamino-2-hydroxypropane, N-methyl-1,2-diaminoethane, 1,4-diazacyclohexane, 1,2-diamino-1,1-dimethylethane, 2,3-diaminobutane, 1,4-diaminobutane, N-hydroxyethyl-1,2-diaminoethane, 1-ethyl-1,3-diaminopropane, 2,2-dimethyl-1,3-diaminopropane, 1,5-diaminopentane, 2-methyl-1,5-diaminopentane, 2,3-diamino-2,3-dimethylbutane, N-2-aminoethylmorpholine, 1,6-diaminohexane, 1,6-diamino-2,2,4-trimethylhexane, N,N-dihydroxyethyl-1,2-diaminoethane, N,N-dimethyl-1,2-diamino-ethane, 4,9-dioxa-1,12-diaminododecane, 1,2-diaminocyclohexane, 1,3-diamino-4-methyl-cyclohexane, 1,2-diaminocyclohexane, 1-amino-2-aminomethyl-2-methyl-4,4-dimethyl-cyclohexane, 1,3-diaminomethylcyclohexane, N-2-aminoethylpiperazine, 1,1-di(4-amino-cyclohexyl)methane, 1,1-di(4-aminophenyl)methane, N,N′-di-isopropyl-p-phenylenediamine, N,N′-di-sec-butyl-p-phenylenediamine, N,N′-bis(1,4-dimethyl-pentyl)-p-phenylenediamine, N,N′-bis(1-ethyl-3-methyl-pentyl)-p-phenylenediamine, N,N′-bis(1-methyl-heptyl)-p-phenylene-diamine, N,N′-dicyclohexyl-p-phenylenediamine, N,N′-diphenyl-p-phenylenediamine, N,N′-di-(2-naphthyl)-p-phenylenediamine, N-isopropyl-N′-phenyl-p-phenylenediamine, N-(1,3-dimethyl-butyl)-N′-phenyl-p-phenylenediamine, N-(1-methyl-heptyl)-N′-phenyl-p-phenylenediamine, N-cyclohexyl-N′-phenyl-p-phenylenediamine and N,N′-dimethyl-N,N′-di-sec-butyl-p-phenylene-diamine.

In the groups —OR¹, —SR², —C(O)OR³ and —C(O)R⁴, R¹, R², R³ and R⁴ may have the following meanings, inter alia:

R¹, R², R³ and R⁴ in the meaning of C₁₋₄alkyl are, for example, methyl, ethyl, n-propyl, isopropyl, n-butyl, isobutyl, sec-butyl or tert-butyl, preferably methyl and ethyl, and in the meaning of C₆₋₁₂aryl are phenyl, biphenyl or naphthyl, preferably phenyl.

In the group —NR⁵R⁶, R⁵ and R⁶, in addition to being a hydrogen atom, are a C₁₋₄alkyl radical, for example methyl, ethyl, n-propyl, isopropyl, n-butyl, isobutyl, sec-butyl or tert-butyl, preferably methyl and ethyl, or a radical —(CH₂)ₒH, wherein o is an integer from 1 to 6, especially 2 or 3, and the nitrogen atom is preferably symmetrically substituted.

Greatest preference is given to E being selected from the following groups:

a hydrogen atom, —(CH₂)ₑ-E′ and

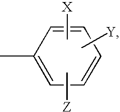

wherein e is an integer from 1 to 6, especially 2 and 3, E′ is a hydrogen atom, a group —OH, —OCat, —SH, —SCat, —OR¹, —SR², —NR⁵R⁶ or —C(O)OR³, and X, Y and Z are selected each independently of the others from a hydrogen atom and an —OH, —OCat, —SH, —SCat, —OR¹, —SR², —NR⁵R⁶ and —C(O)OR³ group, R¹, R² and R³ being each independently of the others a C₁₋₄alkyl radical, especially methyl or ethyl, and R⁵ and R⁶ being a radical —(CH₂)ₒOH, wherein o is an integer from 2 to 6, and Cat is a sodium or potassium cation or unsubstituted ammonium or an ammonium cation described hereinbefore as being preferred, or
is selected from groups of the following formulae

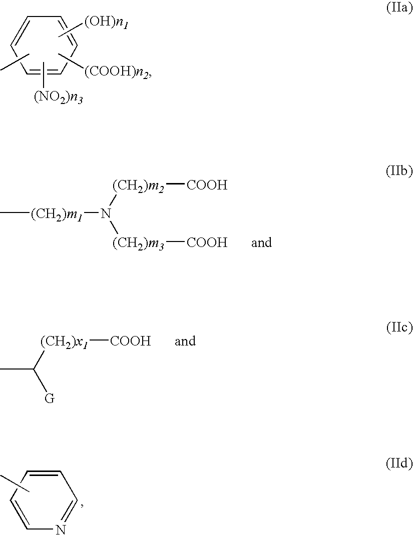

wherein
n₁ and n₂ are each independently of the other 0, 1 or 2, at least one group —OH or —COOH being present, and n₃ is 0 or 1,
m₁ is an integer from 1 to 8,
m₂ and m₃ are each independently of the other an integer from 1 to 8,
G is a group —NH₂, —OH, —COOH or —SO₃H, and
x₁ is an integer from 0 to 8.
D is the radical of known chromophores having the basic structure D(H)ₘ₊ₙ. Examples of such chromophores are described, for example, in W. Herbst, K. Hunger, Industrielle Organische Pigmente, 2nd completely revised edition, VCH 1995. In principle, any chromophore whose basic structure can be modified with one or more sulfonamide groups is suitable. The chromophore is usually selected from the 1-aminoanthraquinone, anthraquinone, anthrapyrimidine, azo, azomethine, benzodifuranone, quinacridone, quinacridone quinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone and thioindigo series.

Examples of pigments (and also substituted derivatives thereof) that may be used as starting compounds for the sulfonamide salts according to the invention and that fall within the above-mentioned pigment classes are described in W. Herbst, K. Hunger, Industrielle Organische Pigmente, 2nd completely revised edition, VCH 1995: 1-aminoanthraquinone pigments: p. 503-511; anthraquinone pigments: p. 504-506, 513-521 and 521-530; anthrapyrimidine: p. 513-415; azo pigments: p. 219-324 and 380-398; azomethine pigments: p. 402-411; quinacridone pigments: p. 462-481; quinacridone quinone pigments: p. 467-468; quinophthalone pigments: p. 567-570; diketopyrrolopyrrole pigments: p. 570-574; dioxazine pigments: p. 531-538; flavanthrone pigments: p. 517-519, 521; indanthrone pigments: p. 515-517; isoindoline pigments: p. 413-429; isoindolinone pigments: p. 413-429; isoviolanthrone pigments: p. 528-530; perinone pigments: p. 482-492; perylene pigments: p. 482-496; phthalocyanine pigments: p. 431-460; pyranthrone pigments: p. 522-526; thioindigo pigments (indigo pigments): p. 497-500, it also being possible to use mixtures of such pigments, including solid solutions.

Among the colorants of formula II preference is given to the following, E being as defined hereinbefore:
1-aminoanthraquinones or anthraquinones of formula

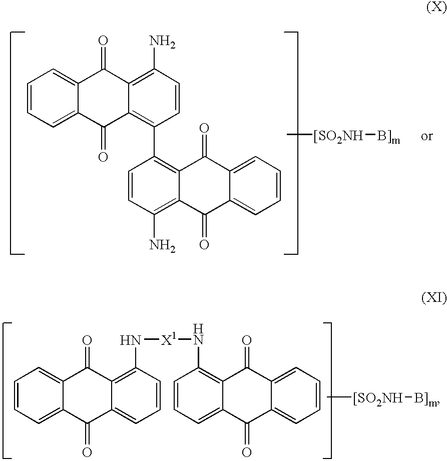

wherein X¹ is a group

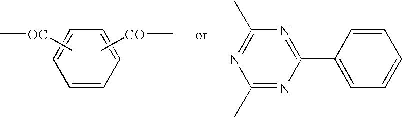

and m has a value from 1 to 4, preferably from 2 to 3;
quinacridones of formula

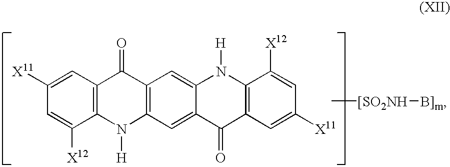

wherein X¹¹ and X¹² are each independently of the other hydrogen, halogen, C₁-C₂₄alkyl, C₁-C₆alkoxy or phenyl and m has a value from 1 to 4, especially from 2 to 3;
pyrrolo[3,4-c]pyrroles of formula

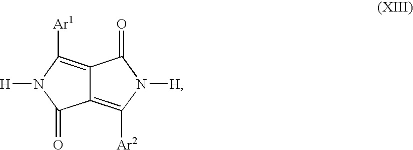

wherein Ar¹ and Ar² are each independently of the other a group of formula

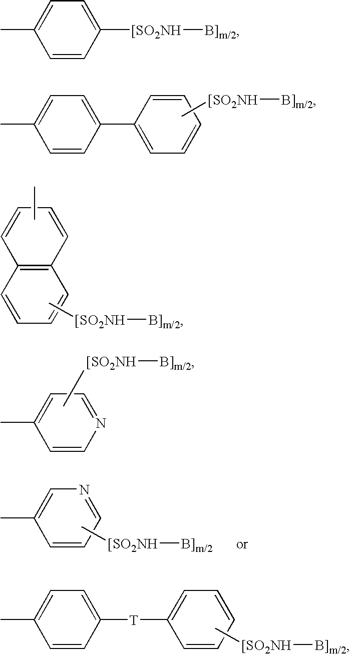

wherein T is —CH₂—, —CH(CH₃)—, —C(CH₃)₂—, —CH═N—, —N═N—, —O—, —S—, —SO—, —SO₂— or —NX¹³—, X¹³ being hydrogen or C₁₋₆-alkyl, especially methyl or ethyl, and m has a value from 1 to 4, especially from 2 to 3,
dioxazines of formula

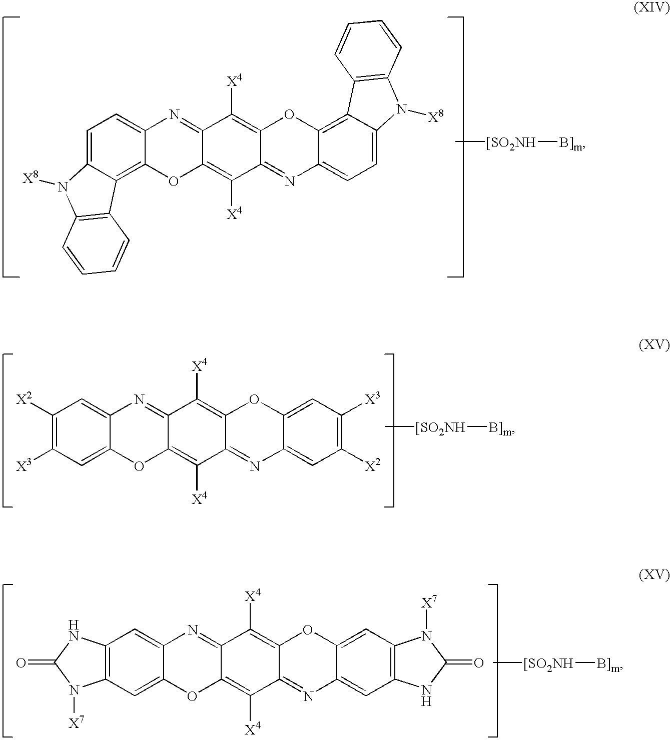

wherein X² is a C₁₋₄alkoxy radical, especially ethoxy, X³ is a C₁₋₄acylamino, especially acetylamino, or benzoylamino group and X⁴ is a chlorine atom or a radical NHC(O)CH₃, X⁷ is a hydrogen atom, a C₁₋₈alkyl radical, a substituted or unsubstituted phenyl, benzyl, benzanilide or naphthyl group, a C₅₋₇cycloalkyl radical or a radical of formula

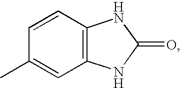

X⁸ is a hydrogen atom or a C₁₋₄alkyl radical, and m has a value from 1 to 4;
flavanthrones of formula

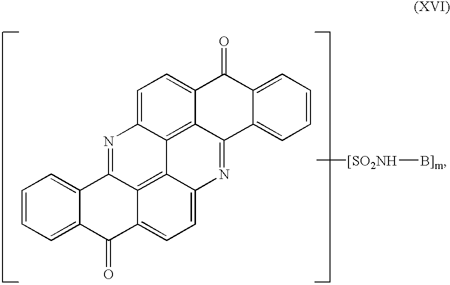

wherein m has a value from 1 to 4, preferably from 2 to 3;
indanthrones of formula

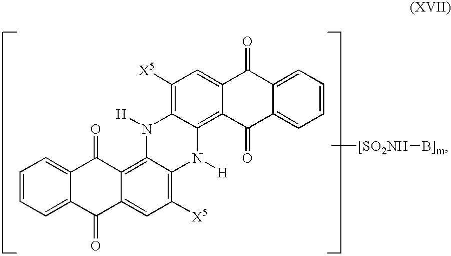

wherein X⁵ is a hydrogen or chlorine atom, and m has a value from 1 to 4, preferably from 2 to 3;
indigo derivatives of formula

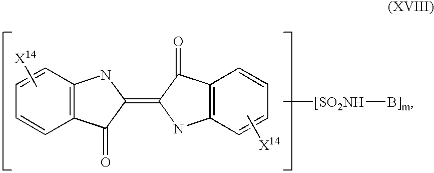

wherein X¹⁴ is hydrogen, CN, C₁₋₆-alkyl, C₁₋₆alkoxy or halogen, and m has a value from 1 to 3;
isoviolanthrones of formula

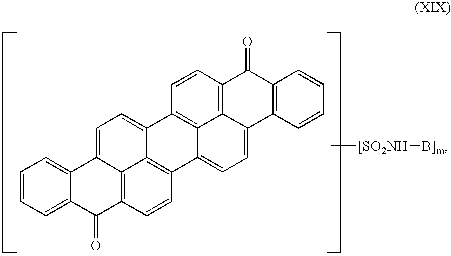

wherein m has a value from 1 to 4;
perinone pigments of formula

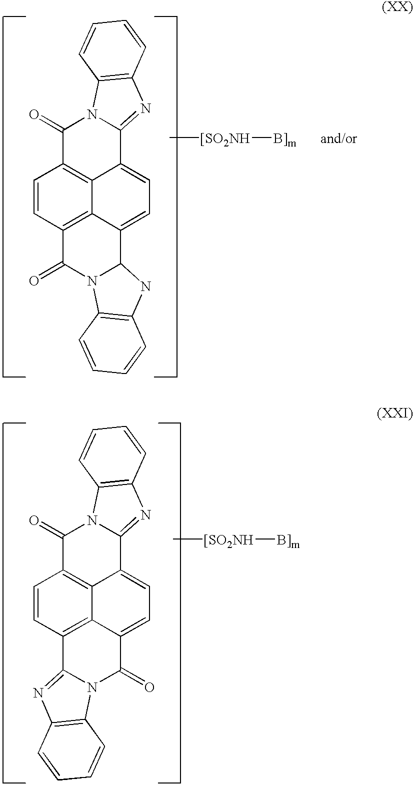

wherein m has a value from 1 to 4;
perylenes of formula

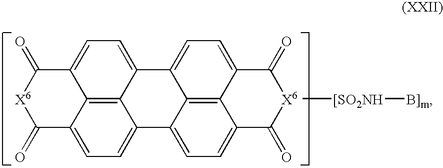

wherein X⁶ is O or NX¹⁵, X¹⁵ being hydrogen, CH₃ or unsubstituted or substituted phenyl or C₇₋₁₁aralkyl, such as benzyl or 2-phenylethyl, and m has a value of from 1 to 4, especially from 2 to 3, it being possible for the phenyl ring to be substituted by methyl, methoxy, ethoxy or —N═N-Ph;
phthalocyanines of formula

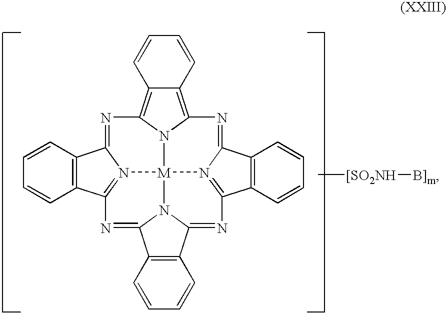

wherein M is H₂, a bivalent metal selected from the group Cu(II), Zn(II), Fe(II), Ni(II), Ru(II), Rh(II), Pd(II), Pt(II), Mn(II), Mg(II), Be(II), Ca(II), Ba(II), Cd(II), Hg(II), Sn(II), Co(II) and Pb(II), or a divalent oxometal selected from the group V(O), Mn(O) and TiO, and m has a value from 2 to 6, especially from 3 to 5;
pyranthrones of formula

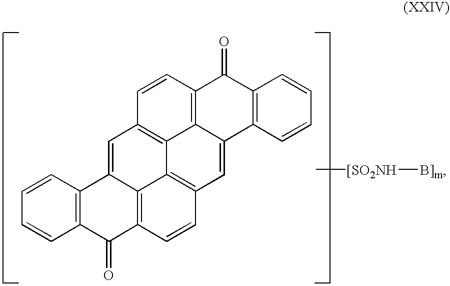

and derivatives of the basic structure halogenated with bromine or chlorine or bromine and chlorine, for example the 2,10-dichloro, 4,6- and 6,14-dibromo derivatives, m having a value from 2 to 4,
thioindigo derivatives of formula

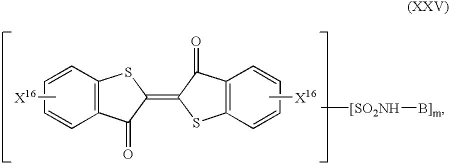

wherein X¹⁶ is hydrogen, CN, C₁₋₆alkyl, especially methyl, C₁₋₆alkoxy, especially methoxy, or halogen, especially chlorine, and m has a value from 1 to 3,
monoazo yellow and orange pigments of formula

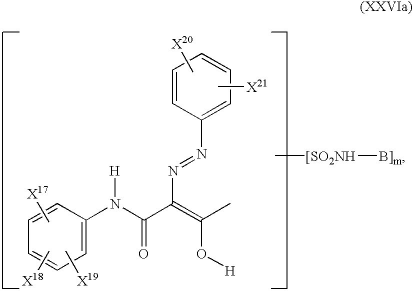

wherein m has a value from 1 to 4,
diaryl yellow pigments of formula

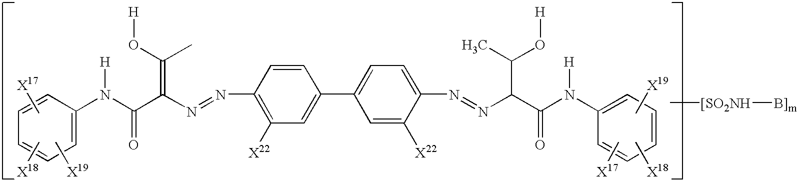

(XXVIb), wherein m has a value from 2 to 4,
naphthol AS pigments of formula

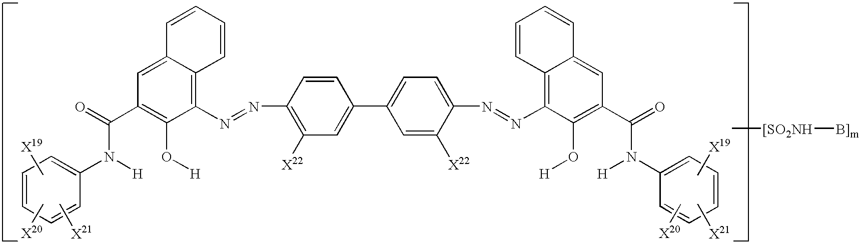

(XXVIc), wherein m has a value from 2 to 6,
X¹⁷ to X²¹: are each independently of the other a hydrogen atom, a halogen atom, C₁₋₆-alkyl, C₁₋₆alkoxy, a nitro group or an acetyl group,
X²² is a hydrogen atom, a halogen atom, C₁₋₆alkyl or C₁₋₆alkoxy,
monoazo quinolone pigments of formula

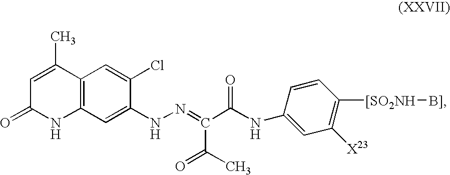

wherein X²³ is hydrogen, halogen, C₁₋₄alkyl, C₁₋₄alkoxycarbonyl, C₁₋₄alkylcarbonyl, C₁₋₄-alkanoylamino (preparation described in WO 02/34839), and
azo pigments of formula

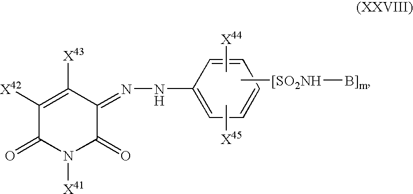

wherein

X⁴¹ is a hydrogen atom, a C₁₋₄alkyl radical, such as methyl or ethyl, or a perfluoro-C₁₋₄alkyl radical, such as trifluoromethyl, a hydroxy-C₁₋₄alkyl radical, or a C₁₋₈alkyl radical interrupted one or more times by —O—, such as CH₂CH₂CH₂—O—CH(CH₃)₂, a C₆₋₁₀aryl radical, such as phenyl, or a C₇₋₁₂aralkyl radical, such as benzyl,

X⁴² is a hydrogen atom, a cyano group or a carboxamide group,
X⁴³ is a hydrogen atom, a carboxylic acid group or a salt thereof, or a C₁₋₄alkyl radical,
X⁴⁴ and X⁴⁵ are a C₁₋₄alkyl radical, such as methyl or ethyl, a perfluoro-C₁₋₄alkyl radical, such as trifluoromethyl, an C₁₋₄alkoxy radical, such as methoxy or ethoxy, a nitro group, a halogen atom, such as chlorine, COOX⁴⁶ (X⁴⁶ being a C₁₋₄alkyl radical, a C₆₋₁₀aryl radical which is unsubstituted or substituted by, for example, 1 or 2 chlorine atoms, such as phenyl or 1,4-dichlorophenyl, or a C₇₋₁₂aralkyl radical, such as benzyl), CONHX⁴⁷, X⁴⁷ being a C₁₋₄alkyl radical, a C₆₋₁₀aryl radical, such as phenyl, or a C₇₋₁₂aralkyl radical, such as benzyl, and m has a value from 1 to 2,
isoindoline pigments of formula

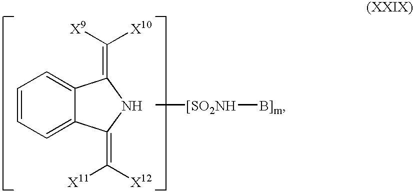

wherein X⁹, X¹⁰, X¹¹ and X¹² are CN, CONH—C₁₋₈alkyl or CONH—C₆₋₁₀aryl, or X⁹ and X¹⁰ and/or X¹¹ and X¹² are in each case members of a heterocyclic ring, such as

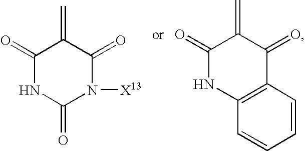

X¹³ being a hydrogen atom or a C₆₋₁₀aryl radical, and m has a value from 1 to 4,
isoindolines of formula

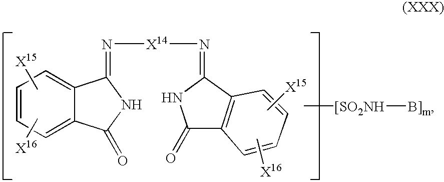

wherein X¹⁴ is the radical of an aromatic or heteroaromatic diamine, such as

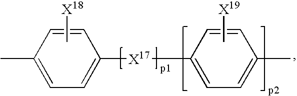

p1 and p2 being 0 or 1, X¹⁸ and X¹⁹ being a hydrogen atom, a C₁₋₄alkyl radical, a C₁₋₄alkoxy radical or a chlorine atom, X¹⁷ being a group —CH₂—, —CH═CH— or —N═N—, X¹⁵ and X¹⁶ are a hydrogen atom, a C₁₋₄alkyl radical, a C₁₋₄alkoxy radical, a nitro group or a chlorine atom, and m has a value from 1 to 3, and salts thereof.

Preferred colorants are:
pyrrolo[3,4-c]pyrroles of formula

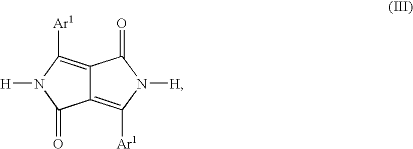

wherein Ar¹ is a group of formula

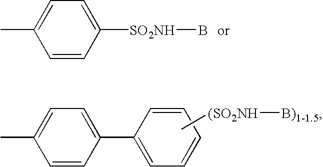

phthalocyanines of formula

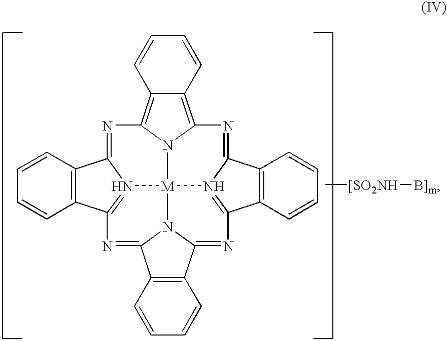

wherein M is Cu(II) or Zn(II), and m has a value from 3 to 5,
indanthrone derivatives of formula

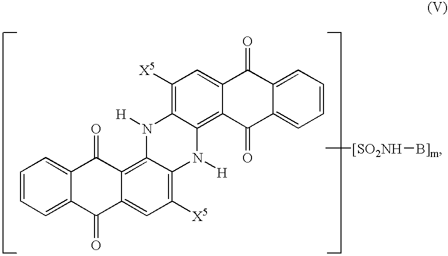

wherein X⁵ is a hydrogen or chlorine atom, and m has a value from 2 to 4, and
quinacridones of formula

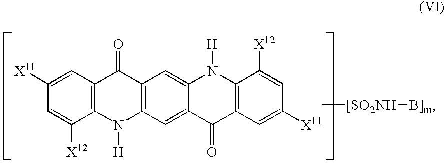

wherein X¹¹ and X¹² are each independently of the other hydrogen, a chlorine atom or a methyl group, m has a value from 1 to 4, and
B is a group mentioned hereinbefore as being preferred for E, and salts thereof.

The colorants of formula II are derived especially from C. I. Pigment Yellow 138, 139, 185, C.I. Pigment Brown 38, C.I. Pigment Orange 66, 69, C.I. Pigment Red 260; C. I. Pigment Red 123, 149, 178, 179, 190, 224, C.I. Pigment Violet 29, C.I. Pigment Black 31, 32; C.I. Pigment Blue 15:6; C.I. Pigment Violet 19, C.I. Pigment Red 122, 192, 202, 207 and 209; C.I. Pigment Red 254, C.I. Pigment Red 255, C.I. Pigment Red 264, C.I. Pigment Red 272, C.I. Pigment Orange 71, C.I. Pigment Orange 73; C.I. Pigment Blue 60 and 64; C.I. Pigment Violet 29, C.I. Pigment Red 123, 179, 190 or C.I. Pigment Violet 23 or 37.

The colorants of formula II according to the invention can be obtained from the corresponding sulfonamides by reaction with alkali metal or ammonium hydroxides or amines. The sulfonamides used as starting materials are either known (see, for example, GB-A-1 198 501, U.S. Pat. No. 4,234,486, U.S. Pat. No. 6,066,203 etc.) or can be prepared using known methods (see, for example, U.S. Pat. No. 6,066,203, 3rd column, lines 36 to 50).

Especially on metallic, non-metallic, metal oxide and (non-metal) oxide surfaces, colorants having selected substituents, which are, for example, from benzoic acid, phthalic acid, isophthalic acid, terephthalic acid, salicylic acid, nitrosalicylic acid, nitrophenol, phenol, dihydroxybenzenes, but also heterocycles, such as pyridine, are suitable especially for chelate formation and adhesion promotion. Chelation of this kind may, in addition, result in the metal particles being protected from corrosion. Aromatic derivatives are especially suitable as (sulfonamide) substituents because they are able to bring about additional π-π stabilisation.

Preference is accordingly given to the use of colorants of formula II

D(SO₂NHE)_{y} (II),

wherein
Y and D are as defined hereinbefore, and
E is selected from groups of the following formulae

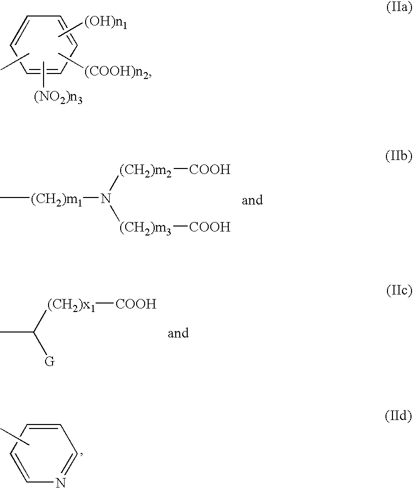

wherein
n₁ and n₂ are each independently of the other 0, 1 or 2, at least one group —OH or —COOH being present, n₃ is 0 or 1,
m₁ is an integer from 1 to 8,
m₂ and m₃ are each independently of the other an integer from 1 to 8,
G is a group —NH₂, —OH, —COOH or —SO₃H, and
x₁ is an integer from 0 to 8. Those colorants and salts thereof in which the hydrogen atoms of the sulfonamide, carboxylic acid and/or hydroxy group have been replaced by an alkali metal cation or ammonium cation are novel and the present invention relates also thereto. These novel colorants may, as described in EP02/04071, be used in the pigmenting of porous materials and in ink-jet printing.

In a preferred embodiment, the colorant, for example tetrakis(sulfon-4-aminosalicylic acid) copper phthalocyanine, is dissolved at room temperature in water, adding as much sodium hydroxide solution as is required to reach a pH of from 8 to 12. The carrier particles, for example aluminium flakes (Eckart Standard 3010) are stirred into the resulting solution at from 0° C. up to 50° C., preferably at room temperature, and mixed thoroughly. The alkaline dispersion is then acidified to a pH from 1.5 to 6.5 with hydrochloric acid, with vigorous stirring, whereupon the colour of the solution changes markedly. The coloured substrate particles are, in customary manner, filtered off, washed and dried. The product, which has a metallic blue shimmer, obtained in the case of tetrakis(sulfon-4-aminosalicylic acid) copper phthalocyanine can be incorporated into plastics and surface-coating compositions without further after-treatment and yields a blue metallic effect.

In like manner it is also possible for pearlescent pigments, such as, for example Iriodin® 9103 Sterling. Silver WR, to be coloured lastingly and with good light-fastness properties. In the case of tetrakis(sulfon-4-aminosalicylic acid) copper phthalocyanine, this results in a product which has a silvery-blue shimmer and which can be incorporated into plastics and surface-coating compositions without further after-treatment and yields a blue silver pearlescent effect.

The present invention relates also to a method in which SiO₂ or SiO₂ and a pigment are applied by precipitation at the same time as the colorant.

In a preferred embodiment, the colorant, for example tetrakis(sulfon-4-aminosalicylic acid) copper phthalocyanine, is dissolved at room temperature in water, adding sodium silicate (soda waterglass) and sodium hydroxide solution until a pH of from 8 to 12 has been reached. The substrate particles, for example aluminium flakes (Eckart Aloxal 3010) are stirred into the resulting solution at from 0 to 30° C., preferably at room temperature, and mixed thoroughly. The alkaline dispersion is acidified to pH 3.5, whereupon the colour of the solution changes markedly. The substrate particles are isolated in customary manner, for example by filtering off, washing and drying. The product, which has a metallic blue shimmer, obtained in the case of tetrakis(sulfon-4-aminosalicylic acid) copper phthalocyanine can be incorporated into plastics and surface-coating compositions without further after-treatment and yields a blue metallic effect.

According to the method described hereinbefore, in addition to the deposition of SiO₂ and colorant or SiO₂, colorant and pigment, it is also possible for dispersed pigment particles and SiO₂ to be deposited onto substrates.

The present invention accordingly relates also to a method of producing coloured carrier particles, which comprises

a) dispersing a pigment in an aqueous solution, preferably water,
b) adding soda waterglass,
c) precipitating SiO₂ and the pigment onto the carrier particles by lowering the pH value, and also to the carrier particles obtainable in accordance with the method.

The method is carried out in principle as described hereinbefore for the colorant. Where appropriate, in Step b) the pH can be adjusted to a value from 7 to 9 by adding a base to the pigment/soda waterglass dispersion, and the pigment and SiO₂ can be precipitated onto the carrier particles by lowering the pH to a value less than 7. Any desired base can be used in the method. Preference is given to alkali metal hydroxides, especially sodium hydroxide. Lowering of the pH is carried out by adding acid, it being possible in principle to use any acid. Preference is given to hydrochloric acid. The pigments are generally selected from 1-aminoanthraquinone, anthraquinone, anthrapyrimidine, azo, azomethine, benzodifuranone, quinacridone, quinacridone quinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone and thioindigo pigments.

SiO₂ can also be precipitated in analogy to a method described in DE-A-195 01 307, by producing the silicon oxide layer by means of a sol-gel process by controlled hydrolysis of one or more metal acid esters in the presence of one or more of the colorants according to the invention and, optionally, an organic solvent and, optionally, a basic catalyst.

Suitable basic catalysts are, for example, amines, such as triethylamine, ethylenediamine, tributylamine, dimethylethanolamine and methoxypropylamine.

The organic solvent is a water-miscible organic solvent, such as a C₁₋₄alcohol, especially isopropanol.

Suitable metal acid esters are selected from alkyl- and aryl-alcoholates, carboxylates, alkyl alcoholates substituted by carboxyl radicals or alkyl radicals or aryl radicals, and carboxylates of silicon. Preference is given to the use of tetraethyl orthosilicate. Furthermore, acetyl-acetonates and acetoacetylacetonates of the afore-mentioned metals may be used.

### Method B (Latent Pigment)

A further embodiment of the present invention relates to a method of producing coloured carrier particles, which comprises

a) dispersing the carrier particles in a solution of a latent pigment, adding the carrier particles to a solution of a latent pigment or adding a latent pigment to a dispersion of the carrier particles,
b) precipitating the latent pigment onto the carrier particles, and
c) subsequently converting to the pigment.

Preference is given to a method which comprises

a) adding carrier particles to a solution of a latent pigment,
b) precipitating the latent pigment onto the carrier particles, and
c) subsequently converting the latent pigment to the pigment.

Method B utilises the good solubility of “latent pigments” in organic solvents. The solubility of the actual pigments, for example diketopyrrolopyrroles (DPPs), is too low in certain selected solvents and/or deposition is unsuccessful because of inadequate adhesion of the pigment to the substrate. The pigment particles produced on the substrate surface according to Method B exhibit good adhesion and light-fastness.

The latent pigment generally has the following formula A(B)ₓ (I) wherein

x is an integer from 1 to 8,
A is the radical of a chromophore of the quinacridone, anthraquinone, perylene, indigo, quinophthalone, indanthrone, isoindolinone, isoindoline, dioxazine, azo, phthalocyanine or diketopyrrolopyrrole series, which is linked to x groups B by one or more hetero atoms, those hetero atoms being selected from the group consisting of nitrogen, oxygen and sulfur and forming part of the radical A,
B is a group of the formula

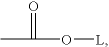

it being possible for the groups B, when x is a number from 2 to 8, to be the same or different, and
L is any desired group suitable for imparting solubility.

L is preferably a group of formula

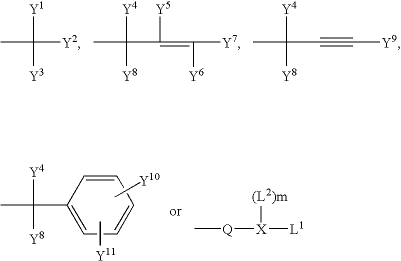

wherein Y¹, Y² and Y³ are each independently of the others C₁-C₆alkyl,
Y⁴ and Y⁸ are each independently of the other C₁-C₆alkyl, C₁-C₆alkyl interrupted by oxygen, sulfur or N(Y¹²)₂, or unsubstituted or C₁-C₆alkyl-, C₁-C₆alkoxy-, halo-, cyano- or nitro-substituted phenyl or biphenyl,
Y⁵, Y⁶ and Y⁷ are each independently of the others hydrogen or C₁-C₆alkyl, Y⁹ is hydrogen, C₁-C₆alkyl or a group of formula

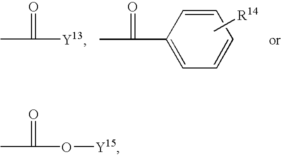

Y¹⁰ and Y¹¹ are each independently of the other hydrogen, C₁-C₆alkyl, C₁-C₆alkoxy, halogen, cyano, nitro, N(Y¹²)₂, or unsubstituted or halo-, cyano-, nitro-, C₁-C₆alkyl- or C₁-C₆alkoxy-substituted phenyl,
Y¹² and Y¹³ are C₁-C₆alkyl, Y¹⁴ is hydrogen or C₁-C₆alkyl, and Y¹⁵ is hydrogen, C₁-C₆alkyl, or unsubstituted or C₁-C₆alkyl-substituted phenyl,
Q is p,q-C₂-C₆alkylene unsubstituted or mono- or poly-substituted by C₁-C₆alkoxy,
C₁-C₆alkylthio or C₂-C₁₂dialkylamino, wherein p and q are different position numbers,
X is a hetero atom selected from the group consisting of nitrogen, oxygen and sulfur, m being the number 0 when X is oxygen or sulfur and m being the number 1 when X is nitrogen, and
L¹ and L² are each independently of the other unsubstituted or mono- or poly-C₁-C₁₂alkoxy-, —C₁-C₁₂alkylthio-, —C₂-C₂₄dialkylamino-, —C₆-C₁₂aryloxy-, —C₆-C₁₂arylthio-, —C₇-C₂₄alkylarylamino- or —C₁₂-C₂₄diarylamino-substituted C₁-C₆alkyl or [-(p′,q′-C₂-C₆alkylene)-Z-]1-C₁-C₆alkyl,
n being a number from 1 to 1000, p′ and q′ being different position numbers, each Z independently of any others being a hetero atom oxygen, sulfur or C₁-C₁₂alkyl-substituted nitrogen, and it being possible for C₂-C₆alkylene in the repeating [-C₂-C₆alkylene-Z-] units to be the same or different,
and L₁ and L₂ may be saturated or unsaturated from once to ten times, may be uninterrupted or interrupted at any location by from 1 to 10 groups selected from the group consisting of —(C═O)— and —C₆H₄—, and may carry no further substituents or from 1 to 10 further substituents selected from the group consisting of halogen, cyano and nitro. Of special interest are compounds of formula (I) wherein L is C₁-C₆alkyl, C₂-C₆alkenyl or

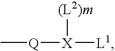

wherein Q is C₂-C₄alkylene, and
L¹ and L² are [-C₂-C₁₂alkylene-Z-]ₙ—C₁-C₁₂alkyl or is C₁-C₁₂alkyl mono- or poly-substituted by C₁-C₁₂alkoxy, C₁-C₁₂alkylthio or C₂-C₂₄dialkylamino, and m and n are as defined hereinbefore. Of very special interest are compounds of formula (I) wherein L is C₄-C₅alkyl, C₃-C₆alkenyl or

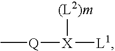

wherein Q is C₂-C₄alkylene, X is oxygen and m is zero, and L¹ is [-C₂-C₁₂alkylene-O-]ₙ—C₁-C₁₂alkyl or is C₁-C₁₂alkyl mono- or poly-substituted by C₁-C₁₂alkoxy, especially those wherein -Q-X— is a group of formula —C(CH₃)₂—CH₂—O—.

Examples of suitable compounds of formula (I) are disclosed in EP-A-0 648 770, EP-A-0 648 817, EP-A-0 742 255, EP-A-0 761 772, WO98/32802, WO98/45757, WO98/58027, WO99/01511, WO00/17275, WO00/39221, WO00/63297 and EP-A-1 086 984. The pigment precursors may be used singly or also in mixtures with other pigment precursors or with colorants, for example customary dyes for the application in question.

A is the radical of known chromophores having the basic structure A(H)ₓ, wherein A preferably has, at each hetero atom linked to x groups B, at least one immediately adjacent or conjugated carbonyl group, such as, for example,

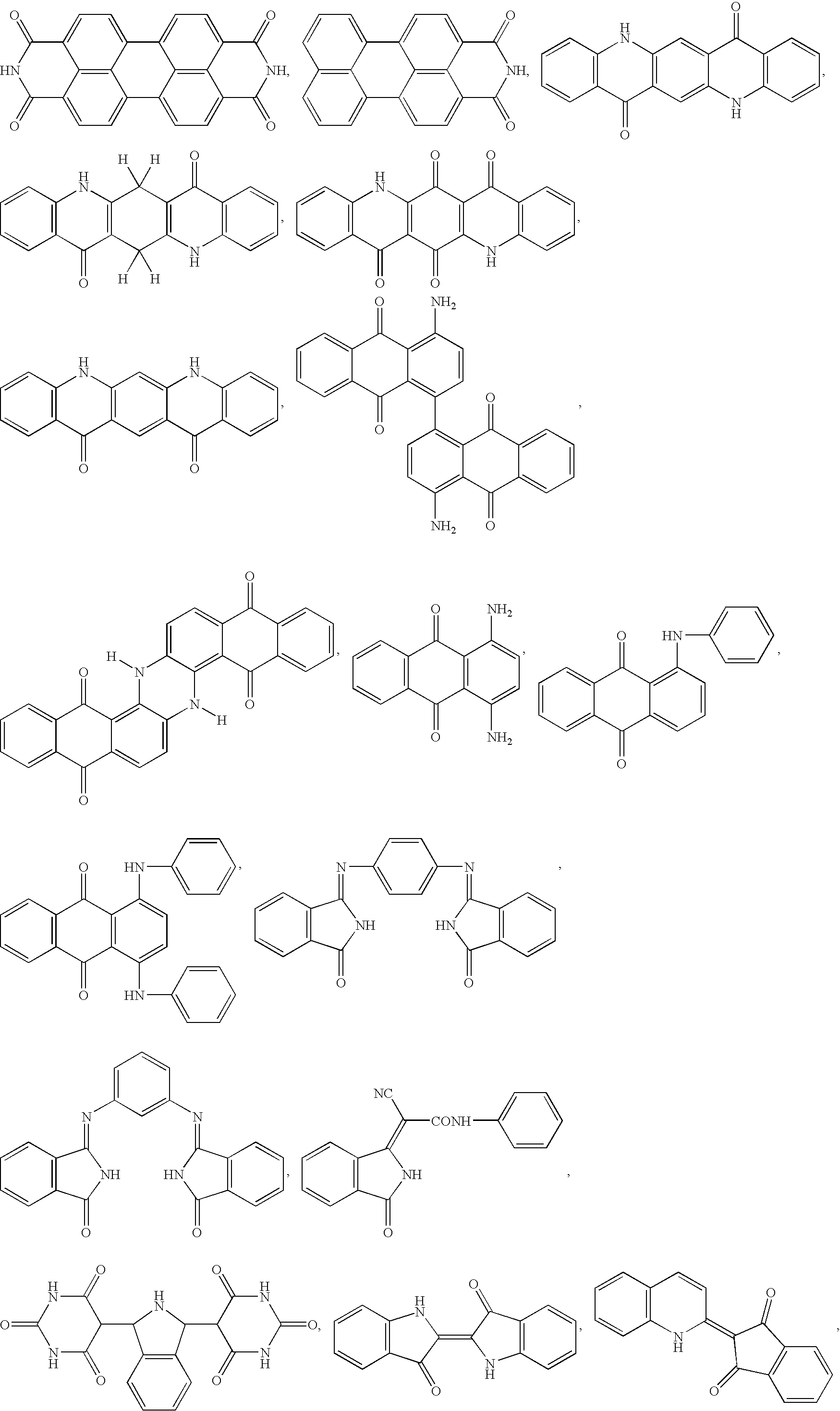

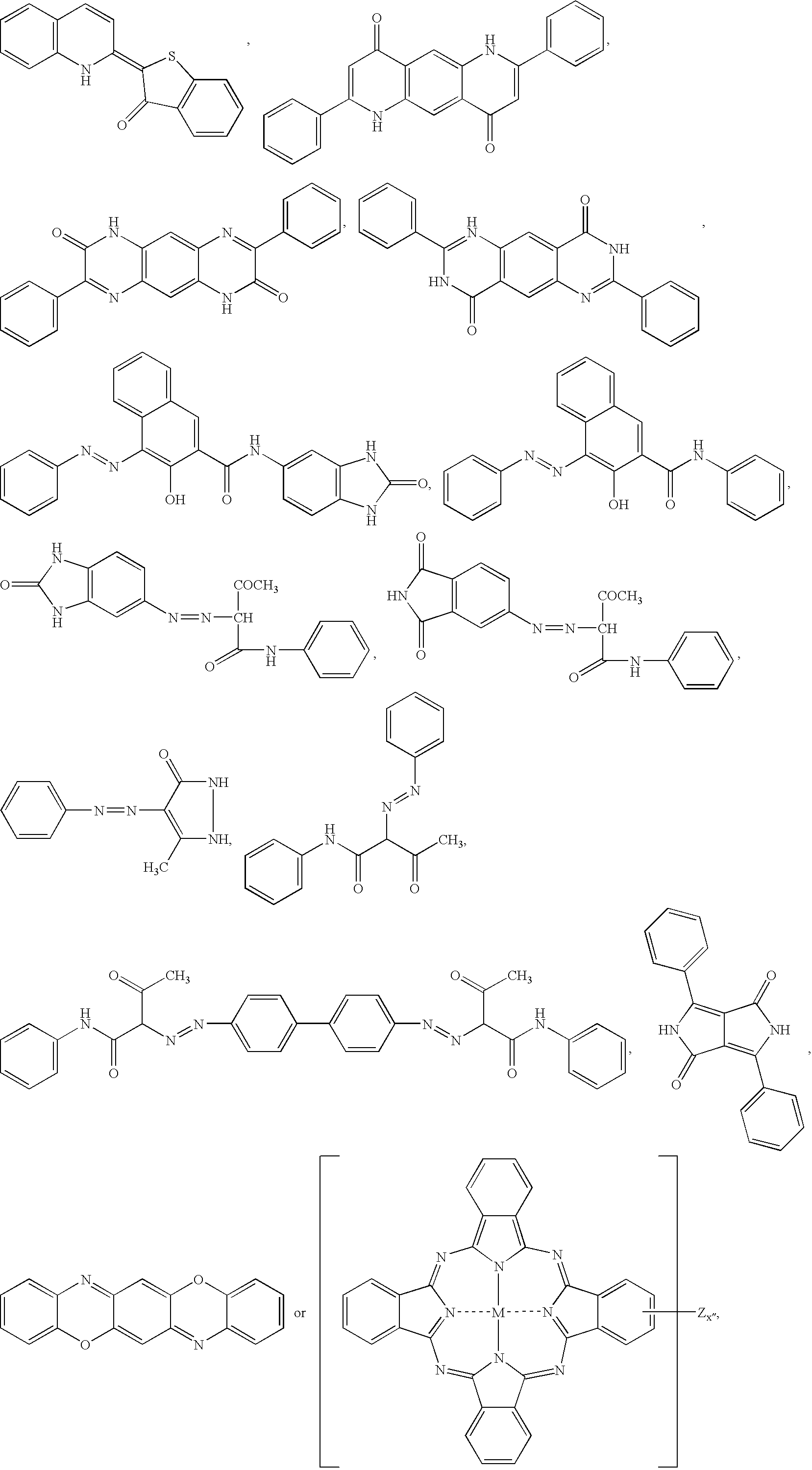

wherein, for example, Z is

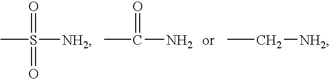

and x″ is a number from 1 to 16, especially from 1 to 4;
and also, in each case, all known derivatives thereof.

Worthy of special mention are those soluble chromophores wherein the pigment of formula A(H)ₓ is Colour Index Pigment Yellow 13, Pigment Yellow 73, Pigment Yellow 74, Pigment Yellow 83, Pigment Yellow 93, Pigment Yellow 94, Pigment Yellow 95, Pigment Yellow 109, Pigment Yellow 110, Pigment Yellow 120, Pigment Yellow 128, Pigment Yellow 139, Pigment Yellow 151, Pigment Yellow 154, Pigment Yellow 175, Pigment Yellow 180, Pigment Yellow 181, Pigment Yellow 185, Pigment Yellow 194, Pigment Orange 31, Pigment Orange 71, Pigment Orange 73, Pigment Red 122, Pigment Red 144, Pigment Red 166, Pigment Red 184, Pigment Red 185, Pigment Red 202, Pigment Red 214, Pigment Red 220, Pigment Red 221, Pigment Red 222, Pigment Red 242, Pigment Red 248, Pigment Red 254, Pigment Red 255, Pigment Red 262, Pigment Red 264, Pigment Brown 23, Pigment Brown 41, Pigment Brown 42, Pigment Blue 25, Pigment Blue 26, Pigment Blue 60, Pigment Blue 64, Pigment Violet 19, Pigment Violet 29, Pigment Violet 32, Pigment Violet 37, 3,6-di(4′-cyano-phenyl)-2,5-dihydro-pyrrolo[3,4-c]pyrrole-1,4-dione, 3,6-di(3,4-dichloro-phenyl)-2,5-dihydro-pyrrolo-[3,4-c]pyrrole-1,4-dione or 3-phenyl-6-(4′-tert-butyl-phenyl)-2,5-dihydro-pyrrolo[3,4-c]pyrrole-1,4-dione. Further examples are described by Willy Herbst and Klaus Hunger in “Industrial Organic Pigments” (ISBN 3-527-28161-4, VCH/Weinheim 1993). In general, those soluble pigment precursors do not have deprotonatable carboxylic acid or sulfonic acid groups.

Alkyl or alkylene may be straight-chained, branched, monocylic or polycyclic. C₁-C₁₂Alkyl is accordingly, for example, methyl, ethyl, n-propyl, isopropyl, n-butyl, sec-butyl, isobutyl, tert-butyl, cyclobutyl, n-pentyl, 2-pentyl, 3-pentyl, 2,2-dimethylpropyl, cyclopentyl, cyclohexyl, n-hexyl, n-octyl, 1,1,3,3-tetramethylbutyl, 2-ethylhexyl, nonyl, trimethylcyclohexyl, decyl, menthyl, thujyl, bornyl, 1-adamantyl, 2-adamantyl or dodecyl.

When C₂-C₁₂alkyl is mono- or poly-unsaturated, it is C₂-C₁₂alkenyl, C₂-C₁₂alkynyl, C₂-C₁₂alkapolyenyl or C₂-C₁₂alkapolyynyl, it being possible for two or more double bonds to be, where appropriate, isolated or conjugated, such as, for example, vinyl, allyl, 2-propen-2-yl, 2-buten-1-yl, 3-buten-1-yl, 1,3-butadien-2-yl, 2-cyclobuten-1-yl, 2-penten-1-yl, 3-penten-2-yl, 2-methyl-1-buten-3-yl, 2-methyl-3-buten-2-yl, 3-methyl-2-buten-1-yl, 1,4-pentadien-3-yl, 2-cyclopenten-1-yl, 2-cyclohexen-1-yl, 3-cyclohexen-1-yl, 2,4-cyclohexadien-1-yl, 1-p-menthen-8-yl, 4(10)-thujen-10-yl, 2-norbornen-1-yl, 2,5-norbornadien-1-yl, 7,7-dimethyl-2,4-norcaradien-3-yl and the various isomers of hexenyl, octenyl, nonenyl, decenyl and dodecenyl. C₂-C₄Alkylene is, for example, 1,2-ethylene, 1,2-propylene, 1,3-propylene, 1,2-butylene, 1,3-butylene, 2,3-butylene, 1,4-butylene and 2-methyl-1,2-propylene. C₅-C₁₂Alkylene is, for example, an isomer of pentylene, hexylene, octylene, decylene or dodecylene.

C₁-C₁₂Alkoxy is O—C₁-C₁₂alkyl, preferably O—C₁-C₄alkyl.

C₆-C₁₂Aryloxy is O—C₆-C₁₂aryl, for example phenoxy or naphthyloxy, preferably phenoxy.

C₁-C₁₂Alkylthio is S—C₁-C₁₂alkyl, preferably S—C₁-C₄alkyl.

C₆-C₁₂Arylthio is S—C₆-C₁₂aryl, for example phenylthio or naphthylthio, preferably phenylthio.

C₂-C₂₄-Dialkylamino is N(alkyl₁)(alkyl₂), the sum of the carbon atoms in the two groups alkyl₁ and alkyl₂ being from 2 to 24, preferably N(C₁-C₄alkyl)C₁-C₄alkyl.

C₇-C₂₄Alkylarylamino is N(alkyl₁)(aryl₂), the sum of the carbon atoms in the two groups alkyl, and aryl₂ being from 7 to 24, for example methylphenylamino, ethylnaphthylamino or butylphenanthrylamino, preferably methylphenylamino or ethylphenylamino.

C₁₂-C₂₄-Diarylamino is N(aryl₁)(aryl₂), the sum of the carbon atoms in the two groups aryl, and aryl₂ being from 12 to 24, for example diphenylamino or phenylnaphthylamino, preferably diphenylamino.

Halogen is chlorine, bromine, fluorine or iodine, preferably fluorine or chlorine, especially chlorine.

The solvent in which the latent pigment of formula I described hereinbefore is soluble is designated hereinafter solvent I. The solvent in which the latent pigment described hereinbefore is sparingly soluble is designated hereinafter solvent II.

In principle, any solvent in which the latent pigment is soluble without undergoing decomposition and in which the substrate remains undissolved is suitable as solvent I. In general, such solvents are organic solvents. Examples include any desired protic or aprotic solvents, such as, for example, hydrocarbons, alcohols, amides, nitriles, nitro compounds, N-heterocyclic compounds, ethers, ketones and esters, which may also be mono- or poly-unsaturated or chlorinated, for example methanol, ethanol, isopropanol, diethyl ether, acetone, methyl ethyl ketone, 1,2-dimethoxyethane, 1,2-diethoxyethane, 2-methoxyethanol, ethyl acetate, tetrahydrofuran, dioxane, acetonitrile, benzonitrile, nitrobenzene, N,N-dimethyl-formamide, N,N-dimethylacetamide, dimethyl sulfoxide, N-methylpyrrolidone, pyridine, picoline, quinoline, trichloroethane, benzene, toluene, xylene, anisole or chlorobenzene. Instead of a single solvent, mixtures of a plurality of solvents may also be used. Preference is given to toluene, methanol, ethanol, isopropanol, 1,2-dimethoxyethane, 1,2-diethoxyethane, 1-methoxy-2-propanol, acetone, methyl ethyl ketone, ethyl acetate, tetrahydrofuran and dioxane, and also to mixtures thereof.

Advantageously, the procedure is such that the latent pigment is first dissolved in a suitable solvent (I) and then the substrate particles are dispersed in the resulting solution. It is, however, also possible, vice versa, for the substrate particles first to be dispersed in the solvent (I) and then for the latent pigment to be added and dissolved.

Any solvent that is miscible with the first solvent and that so reduces the solubility of the pigment that it is completely, or almost completely, deposited onto the substrate is suitable as solvent (II). In this instance, both inorganic solvents and also organic solvents come into consideration. Preference is given to the use of water.

Solvent (II) is slowly added dropwise to the dispersion of the substrate particles or the dispersion is poured into solvent (II). Isolation of the coated substrate can then be carried out in conventional manner by filtering off, washing and drying.

The concentration of the pigment precursor (latent pigment) in water or a solvent is usually from 0.01% by weight to about 99% of the saturation concentration, it being possible in some cases also for supersaturated solutions to be used without premature precipitation of the solvate. In the case of many pigment precursors, the optimum concentration is about from ˜0.05 to 10% by weight, often from about 0.1 to 5% by weight of pigment precursor, based on solvent (I).

Conversion of the pigment precursor into its pigmentary form is carried out by means of fragmentation under known conditions, for example thermally, optionally in the presence of an additional catalyst, for example the catalysts described in WO00/36210.

Heating can be carried out by any means, for example by treatment in a thermal oven or by electromagnetic radiation, for example IR or NIR radiation, or microwaves, optionally in the presence of a catalyst. The conditions required for fragmentation are known per se for each class of pigment precursor.

Advantageously, the temperature for converting the soluble pigment precursors into the corresponding pigments is from 40 to 260° C., preferably from 60 to 200° C., especially from 120 to 180° C.

In a preferred embodiment, the latent pigment, for example

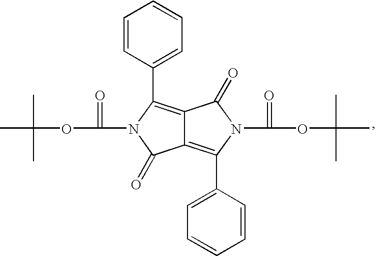

is first completely dissolved in an organic solvent, for example a mixture of THF and ethanol, at a temperature from 20° C. up to the boiling point of the solvent. The solvent is then added to a previously prepared suspension of the carrier particles, for example aluminium flakes (Eckart Standart 3010) or layered silicates, such as, for example, Iriodin Sterling Silver 9103 WR (Merck GmbH & Co. KG) in an organic solvent, for example ethanol, and stirred at a temperature from 20° C. up to the boiling point of the solvent for from 5 to 60 min. Then, within a period of from 10 to 120 min., with vigorous stirring, the solvent in which the latent pigment has poor solubility, normally water, is slowly added dropwise to the mixture, whereupon the latent pigment is deposited onto the carrier particles. Stirring is carried out for a further 10 to 120 min. The carrier particles coloured with the latent pigment are then filtered off, washed and dried.

For converting the latent pigment precipitated onto the aluminium flakes to the pigment, the aluminium flakes are heated, under a protective gas (N₂) or in vacuo, to the temperature required for conversion, for example in the case of the above-mentioned latent pigment derived from C. I. Pigment Red 254 to from 160° C. to 180° C.

The yield is practically quantitative. The effect pigments produced in that manner exhibit good light-fastness properties and no migration (PVC). For the purpose of improving stability, an SiO₂ coating may, where appropriate, be applied to the effect pigments.

Conversely, it is also possible, in the method according to the invention, to use “latent pigments” that are soluble in water/alcohols, preferably in water, for example those that are described in EP-A-1 125 995. Those latent pigments are accordingly precipitated by adding an organic solvent in which the latent pigment has poor solubility. Such water-soluble latent pigments are, for example, latent pigments of formula I wherein B is a group of formula

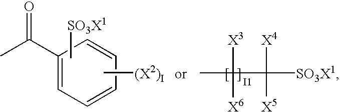

x is a number from 1 to 5, X¹ is a hydrogen atom, an alkali metal cation or an ammonium cation, X² is a substituent, X³, X⁴, X⁵ and X⁶ are a hydrogen atom or a C₁₋₄alkyl radical, I and I1 are a number from 0 to 4, and wherein a plurality of substituents X² may, when I is from 2 to 4, be linked to one another to form a ring.

The coloured (flake-like) carrier particles according to the invention can be used wherever pigments and effect pigments are normally used. An overview of various possibilities for using effect pigments and the compositions used therein is included, for example, in PCT/EP03/01323, PCT/EP03/09296, PCT/EP03/68868, EP 02 405 888.5, EP 02 405 749.9 and EP 02 405 889.3.

The present invention accordingly relates also to the use of the coloured carrier particles for colouring textiles, coating compositions, printing inks, plastics, glass, ceramic products and cosmetic preparations, and also in ink jet printing and to formulations (preparations) for the above-mentioned applications. All customary printing processes can be employed, for example offset printing, intaglio printing, bronzing, flexographic printing.

The pigments of the present invention can be used in admixture with filler pigments, colored and black organic and inorganic pigments, colored and black luster pigments based on, for example, metal oxide coated mica, holographic pigments, liquid crystal polymers (LCPs), or conventional metal pigments.

The concentration of the pigments in the system in which it is to used dependent on the specific application, but is generally between 0.01 and 50% by weight, preferably between 0.1 and 5% by weight, based on the overall solids content of the system.

Plastics comprising the pigment of the invention in amounts of 0.1 to 50% by weight, in particular 0.5 to 7% by weight.

In the coating sector, the pigments of the invention are employed in amounts of 0.5 to 10% by weight.

In the pigmentation of binder systems, for example for paints and printing inks for intaglio, offset or screen printing, the pigment is incorporated into the printing ink in amounts of 2 to 50% by weight, preferably 5 to 30% by weight and in particular 8 to 15% by weight.

The pigments according to the invention are also suitable for making-up the lips or the skin and for colouring the hair or the nails. The invention accordingly relates also to a cosmetic preparation or formulation comprising from 0.0001 to 90% by weight of a pigment, especially an pigment, according to the invention and from 10 to 99.9999% of a cosmetically suitable carrier material, based on the total weight of the cosmetic preparation or formulation. Such cosmetic preparations or formulations are, for example, lipsticks, blushers, foundations, nail varnishes and hair shampoos.

The pigments may be used singly or in the form of mixtures. It is, in addition, possible to use pigments according to the invention together with other pigments and/or colorants, for example in combinations as described hereinbefore or as known in cosmetic preparations. The cosmetic preparations and formulations according to the invention preferably contain the pigment according to the invention in an amount from 0.005 to 50% by weight, based on the total weight of the preparation. Suitable carrier materials for the cosmetic preparations and formulations according to the invention include the customary materials used in such compositions (see, for example, PCT/EP03/0219).

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of sticks, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminium.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation. The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture of such substances. A lipophilic functional cosmetic active ingredient suitable for skin cosmetics, an active ingredient composition or an active ingredient extract is an ingredient or a mixture of ingredients that is approved for dermal or topical application.

The Examples that follow illustrate the present invention, without limiting the scope thereof. Percentages and parts are always percentages by weight and parts by weight, respectively, unless otherwise specified.

### EXAMPLES

### Example 1

1.2 g of tetrakis(sulfon-4-amidosalicylic acid) copper phthalocyanine are dissolved, at room temperature, in 350 ml of water, adding as much sodium hydroxide solution as is required to reach a pH of 11.6 g of aluminium flakes (Eckart Standard 3010) are stirred into the resulting solution, at room temperature, and mixed thoroughly. The alkaline dispersion is then brought to pH 8 over a period of 5 min. using dilute, 2% aqueous hydrochloric acid with vigorous stirring and is neutralised further over a period of 10 min. In the final step of acid addition, acidification to pH 3.5 is carried out slowly, within a period of 15 min., whereupon the colour of the solution changes markedly. The solution is then heated and stirred for 2 hours at 65° C. and is then allowed to cool and, after being subsequently stirred for a further 2 hours, is filtered. The filter cake is rinsed with 0.1% aqueous hydrochloric acid, subjected to suction until dry and then dried in a vacuum drying cabinet at 40° C. under reduced pressure (−50 hPa). The product, which has a metallic blue shimmer, can be incorporated into plastics and surface-coating compositions without further after-treatment and yields a blue metallic effect.

### Example 2

a) 4 g of

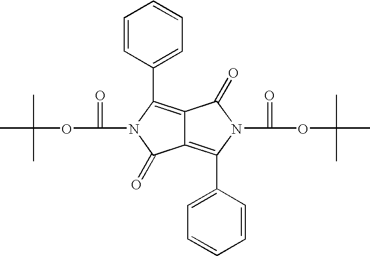

are first completely dissolved in 30 ml of THF and 30 ml of ethanol (94%) at a temperature of 40° C.; the yellow solution is then added to a previously prepared suspension of 15 g of aluminium flakes (Eckart Standart Aloxal 3010) in 150 ml of ethanol (94%) and is stirred at room temperature for 5 min. Then, with vigorous stirring, 300 ml of water are slowly added dropwise to the resulting mixture over a period of 30 min., whereupon the latent pigment is deposited onto the aluminium flakes. The mixture is then stirred for a further 30 min. and is then filtered and washed with 3×200 ml of water, and the filter cake is dried.

For converting the latent pigment into the pigment, the aluminium flakes coated with the latent pigment are heated at 170° C. under a protective gas (N₂) or in vacuo, whereupon the colour of the coating changes from pale-yellow to intense red. The yield is practically quantitative. The effect pigments produced in that manner exhibit good light-fastness properties and no migration (PVC).

b) Example 2a) is repeated, but using, instead of aluminium flakes, the same weight of layered silicates of the type Iriodin® Sterling Silver 9103 WR (Merck GmbH & Co. KG). A pink pearlescent effect pigment having very good light-fastness and weather-fastness properties is obtained.

### Example 3

1.2 g of tetrakis(sulfon-4-amidosalicylic acid) copper phthalocyanine are dissolved, at room temperature, in 350 ml of water, adding as much sodium hydroxide solution as is required to reach a pH of 11.6 g of pearlescent pigment Iriodin® 9103 Sterling Silver WR (Merck GmbH & Co. KG) are stirred into the resulting solution, at room temperature, and mixed thoroughly. The alkaline dispersion is then brought to pH 8 over a period of 5 min. using dilute, 2% aqueous hydrochloric acid with vigorous stirring and is neutralised further over a period of 10 min. In the final step of acid addition, acidification to pH 3.5 is carried out slowly, within a period of 15 min., whereupon the colour of the solution changes markedly. The solution is then heated and stirred for 2 hours at 65° C. and is then allowed to cool and, after being subsequently stirred for a further 2 hours, is filtered. The filter cake is rinsed with 0.5% aqueous hydrochloric acid, subjected to suction until dry and then dried in a vacuum drying cabinet at 40° C. under reduced pressure (˜50 hPa). The product, which has a silvery blue shimmer, can be incorporated into plastics and surface-coating compositions without further after-treatment and yields a blue silver pearlescent effect.

### Example 4

0.34 g of tetrakis(sulfon-4-amidosalicylic acid) copper phthalocyanine are dissolved, at room temperature, in 350 ml of water, adding 1 g of sodium silicate (soda waterglass) and sodium hydroxide until a pH of 10 has been reached. 3.4 g of aluminium flakes (Eckart Aloxal 3010) are stirred into the resulting solution, at room temperature, and mixed thoroughly. The alkaline dispersion is then brought to pH 7 over a period of 5 min. using dilute, 5% aqueous hydrochloric acid with vigorous stirring and is stirred for a further 30 min. In the final step of acid addition, acidification to pH 3.5 is carried out slowly, within a period of 15 min., using 2% HCl, whereupon the colour of the solution changes markedly. The solution is then heated and stirred for 2 hours at 65° C. and is then allowed to cool and, after being subsequently stirred for a further 2 hours, is filtered. The filter cake is rinsed with 0.1% aqueous hydrochloric acid, subjected to suction until dry and then dried in a vacuum drying cabinet at 40° C. under reduced pressure (˜50 hPa). The product, which has a metallic blue shimmer, can be incorporated into plastics and surface-coating compositions without further after-treatment and yields a blue metallic effect.

### Example 5

0.40 g of tetrakis(sulfon-4-amidosalicylic acid) copper phthalocyanine is introduced into a 1000 ml glass beaker with 400.0 g of deionised water and stirred at room temperature. The blue suspension is slowly brought to pH 10.8 at room temperature using 0.30 g of sodium hydroxide solution (32%), whereupon a blue solution is produced. 2.0 g of Aloxal®) 3010 (Eckart-Werke Standard-Bronzepulver-Werke Carl Eckart GmbH & Co.) are introduced and, within a period of 5 min., a homogeneous suspension is produced, with vigorous stirring. Whilst continuing to stir, 0.19 g of aluminium chloride hexahydrate dissolved in 20 g of water is slowly added dropwise, whereupon the blue pigment is deposited onto the aluminium flakes in the form of an aluminium lake and the end pH value is 4.5. The mixture is subsequently stirred for one hour at room temperature and is then heated to 70° C. and stirred vigorously at that temperature for two hours. After stopping the heating, the mixture is stirred for a further hour and is allowed to cool to room temperature.

The coloured aluminium flakes are filtered off, subsequently washed with 100 g of water, and the moist filter cake is dried first for 48 hours in air at room temperature and then at 30° C. and 100 hPa in a vacuum drying cabinet for 8 hours.

2.28 g of blue aluminium flakes having a metal effect are obtained.

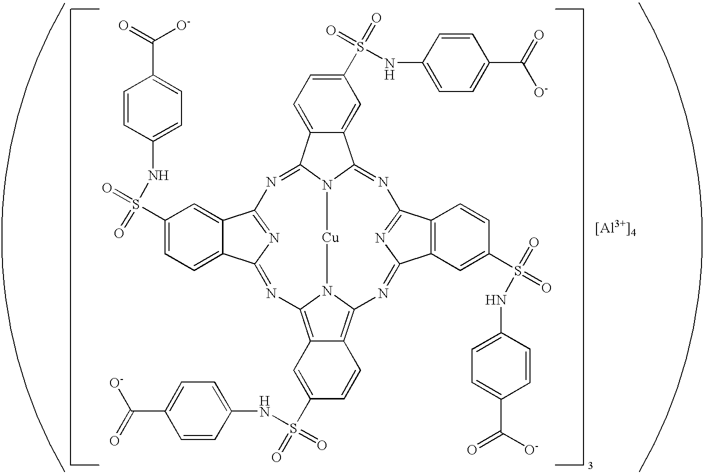

### Synthesis Example 1

### Synthesis of Compound A1′

### Sulfochlorination:

156 g of chlorosulfonic acid are introduced into a 0.5 litre round-bottom flask provided with a stirrer, thermometer and condenser. 30 g of copper phthalocyanine (0.052 mol) are added in portions at room temperature. The resulting solution is slowly heated to 130° C. and stirred for 3 hours. The solution is cooled, and 65.7 g of thionyl chloride are slowly added dropwise at 80° C. Then, for a further 3 hours, stirring under reflux is carried out. At room temperature, the solution is discharged onto 1.3 kg of ice; the resulting suspension is filtered and washed thoroughly with water.

### Amidation:

54 g of 4-aminobenzoic acid (0.393 mol) in 450 g of deionised water and 120 g of methanol are introduced into a 1 litre round-bottom flask and cooled to 0° C. by adding ice. The filter cake, moist with water, is introduced in portions, the temperature being maintained at 0° C. by further addition of ice. The suspension is stirred at 0° C. for 2 hours, at room temperature for 14 hours and at 80° C. for 1 hour. At room temperature, the suspension is filtered and washed with 1000 g of deionised water. After drying in vacuo at 50° C., 95 g of compound A1′ are obtained. Elemental analysis shows that compound A1′ is both a mixture of structural isomers and also a mixture of di- and tri-sulfochlorinated and amidated molecules (ratio: 25% tri-, 75% di-sulfochlorinated species).

### Conversion of Compound A1′ to Compound A1

In a 1500 ml round-bottom flask, 10 g of compound A1′ are suspended in 1000 g of water. At room temperature, 3.7 g of 32% sodium hydroxide solution are added dropwise. The resulting solution is stirred at 60° C. for 2 hours and filtered whilst warm, and the filtrate is concentrated at max. 50° C. and under reduced pressure using a rotary evaporator. After drying in vacuo at 50° C., 10.5 g of compound A1 are obtained.

Elemental analysis (theory): C: 43.71% (49.34%), H, 2.22% (2.21%), N, 10.17% (11.51%), Cu 5.00% (4.35%), Na 7.35% (6.30%), S 9.12% (8.78%).

Analogously to Synthesis Example 1, compounds A2 to A4 are prepared.

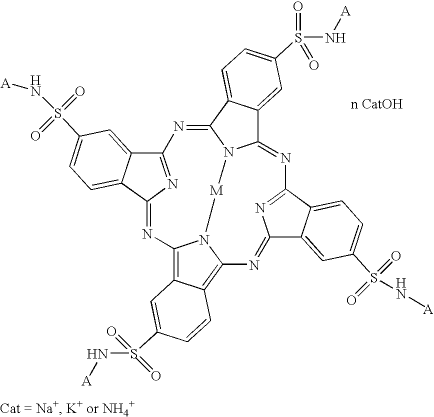

| Synthesis Example | Compound | A | n |
|---|---|---|---|
| 1 | A1 | 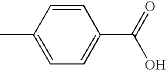 | 1-8 |
| 2 | A2 | 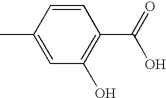 | 1-8 |
| 3 | A3 | 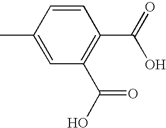 | 1-8 |
| 4 | A4 | 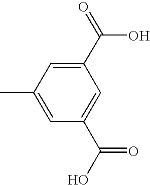 | 1-12 |

### Synthesis Example 5

### Synthesis of Compound B1′

### Sulfochlorination:

222 g of chlorosulfonic acid are introduced into a 0.5 litre round-bottom flask provided with a stirrer, thermometer and condenser. 40 g of C.I. Pigment Red 264 (0.089 mol) are added in portions at room temperature. The resulting solution is slowly heated to 130° C. and stirred for 3 hours. The solution is cooled, and 75.4 g of thionyl chloride are slowly added dropwise at 80° C. Stirring is then carried out at reflux for a further 2 hours. At room temperature, the solution is discharged onto 2.0 g of ice; the resulting suspension is filtered and washed thoroughly with water.

### Amidation:

37 g of 4-aminobenzoic acid (0.266 mol) in 600 g of deionised water and 50 g of methanol are introduced into a 1.5 litre round-bottom flask and cooled to 0° C. by adding ice. The filter cake, moist with water, is introduced in portions, the temperature being maintained at 0° C. by further addition of ice. The suspension is stirred at 0° C. for 2 hours, at room temperature for 14 hours and at 80° C. for 1 hour. At room temperature, the suspension is filtered and washed with 1000 g of deionised water. After drying in vacuo at 50° C., 72 g of compound B1′ are obtained. The ¹H-NMR shows that compound B1′ is both a mixture of structural isomers and also a mixture of di- and tri-sulfochlorinated and amidated molecules (ratio: 25% tri-, 75% di-sulfochlorinated species).

### Conversion of Compound B1′ to Compound B1

In a 1.5 litre round-bottom flask, 10 g of compound B1′ are suspended in 1000 g of water. At room temperature, 3.0 g of 32% sodium hydroxide solution are added dropwise. The resulting solution is stirred at 60° C. for 2 hours and filtered whilst warm, and the filtrate is concentrated by evaporation at max. 50° C. and under reduced pressure. After drying in vacuo at 50° C., 9.7 g of compound B1 are obtained.

Elemental analysis, calculated on the basis of a 1:3 mixture of di- and tri-sulfochlorinated molecules (theory): C: 52.70% (59.86%), H, 3.54% (3.20%), N, 5.41% (6.35%), S: 5.70% (5.21%), Na: 8.61% (7.26%).

Analogously to Synthesis Example 5, compounds B2 and B3 are prepared.

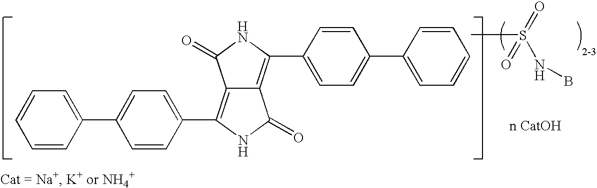

| Synthesis Example | Compound | B | n |
|---|---|---|---|
| 6 | B2 | 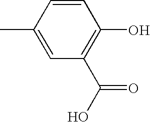 | 1-6 |
| 7 | B3 | 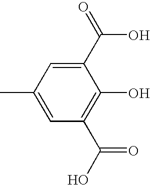 | 1-9 |

### Example 6

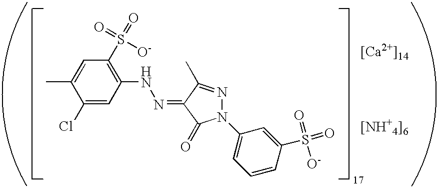

0.40 g of the disulfonic acid of Pigment Yellow 191 is introduced into a 600 ml glass beaker with 100.0 g of deionised water and 1.5 g of sodium silicate, heated to 80° C. and stirred. At 70° C., the orange solution is filtered and introduced into a 600 ml glass beaker and, whilst stirring at room temperature, 1.0 g of Aloxal® 3010 (Eckart GmbH & Co. KG) is introduced and a homogeneous suspension is produced. Using 9.0 g of 2% hydrochloric acid solution, the pH value is quickly brought to 6.0, the dye remaining in solution. Then, 0.11 g of calcium chloride and 0.034 g of ammonium acetate, dissolved in 20 g of water, are slowly added dropwise, whereupon the pH value is 6.4. Using 0.7 g of 2% hydrochloric acid solution, the pH value is slowly lowered to 4.4, whereupon the yellow pigment is deposited onto the aluminium flakes in the form of an NH₄/Ca lake. The mixture is stirred vigorously for 25 min., heated to 70° C. and, at that temperature, stirred for a further hour. With the heating stopped, the mixture is stirred for one hour more and is allowed to cool to room temperature. The pH value is then 4.6.

After filtration and rinsing with 20 g of water, the filter cake is dried first for 48 hours at room temperature and then for 8 hours at 60° C. and 100 hPa in a vacuum drying cabinet. A yellow pigment powder having a metal effect is obtained.

### Example 7

0.50 g of the disulfonic acid of Pigment Yellow 191 is introduced into a 1000 ml glass beaker in 400.0 g of deionised water and heated to 55° C. At that temperature, the orange suspension (pH 4.6) is slowly brought to pH 10.6 using 0.30 g of sodium hydroxide solution (32%), whereupon a clear yellow solution is produced. The heating is stopped and the solution is allowed to cool to 20° C. At that temperature, 2.0 g of Aloxal® 3010 are introduced and, with vigorous stirring over a period of 15 min., a homogeneous suspension is produced. Then, 0.25 g of aluminium chloride hexahydrate, dissolved in 10 g of water, is slowly added dropwise, and the pH value is maintained at 4.5 by adding 0.1 g of 32% sodium hydroxide solution. The yellow pigment is deposited onto the aluminium flakes in the form of an aluminium lake. The mixture is heated to 50° C. and stirred vigorously for one hour. A solution of 0.8 g of calcium chloride in deionised water is then introduced and the pH value is held constant at 5.4. With the heating stopped, the mixture is stirred for one hour more and is allowed to cool to room temperature.

After filtration, the moist filter cake is dried first for 48 hours in air at room temperature and then for 8 hours at 60° C. and 100 hPa in a vacuum drying cabinet, resulting in a green-yellow powder having a metal effect.

### Example 8

Example 7 is repeated except that no CaCl₂ is added. Aluminium flakes are obtained which are coloured with the aluminium salt of the disulfonic acid of Pigment Yellow 191.

### Example 9

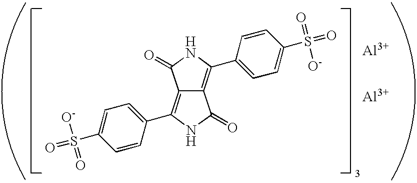

3.00 g of the disulfonic acid sodium salt of Pigment Red 255 are introduced into 1500.00 g of deionised water in a 2000 ml glass beaker, heated to 60° C. and stirred. At 60° C., the pH value of the red solution (pH 4.3) is adjusted to 11.3 using 2.60 g of sodium hydroxide solution (30%). With the heating stopped, stirring is carried out and the red solution is allowed to cool to room temperature. Then 10.00 g of Aloxal® 3010 are introduced and vigorous stirring is carried out. 2.94 g of aluminium chloride hexahydrate, dissolved in 50 g of water, are then slowly added dropwise. The product precipitates out and adheres well to the aluminium flakes. The suspension is heated at 50° C. for one hour, with vigorous stirring. With the heating stopped, stirring is carried out for a further two hours and the mixture is allowed to cool to room temperature. The mixture is filtered using a suction filter, washing being effected with 100.0 g of water. The moist filter cake is dried first for 16 hours in air at room temperature and then for 4 hours at 50° C. and 100 hPa in a vacuum drying cabinet, resulting in a light-red bronze metal effect pigment.

### Example 10

When calcium chloride is used instead of aluminium chloride for precipitation, there are obtained aluminium flakes coloured with the calcium salt of the disulfonic acid of Pigment Red 255.

### Example 11

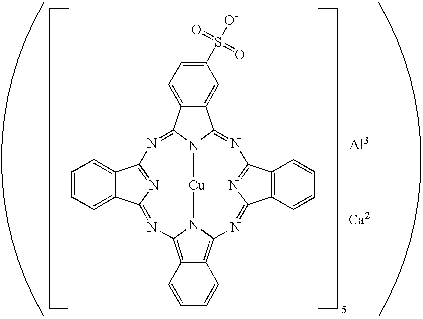

8.30 g of copper phthalocyanine monosulfonic acid (technical grade) are introduced into a 1000 ml glass beaker with 500.00 g of deionised water and 1.60 g of 16% sodium hydroxide solution, stirred and heated at 60° C. for 1 hour. With the heating stopped, further stirring is carried out and the blue solution is allowed to cool to room temperature (pH 11.4). 10.00 g of Aloxal® 3010 are then introduced. Afterwards, 0.254 g of calcium chloride and 0.240 g of aluminium chloride hexahydrate, dissolved in 30 g of water, are slowly added dropwise. The previously dissolved dye precipitates out and adheres to the aluminium flakes. The pH value of the blue suspension is 6.4 and is adjusted to 4.5 using 5.20 g of 2% hydrochloric acid solution. Whilst stirring vigorously, heating at 50° C. is carried out. With the heating stopped, stirring is carried out for a further two hours and the mixture is allowed to cool to room temperature. The pH value is then 4.9 and is brought to 4.5 using 0.30 g of 2% hydrochloric acid solution. The mixture is filtered using a suction filter, washing being effected with 100.0 g of water. The moist filter cake is dried first for 16 hours in air at room temperature and then for 4 hours at 50° C. and 100 hPa in a vacuum drying cabinet, resulting in a blue metal effect pigment.

### Example 12

When Example 11 is repeated and zinc phthalocyanine monosulfonic acid is used instead of copper phthalocyanine monosulfonic acid and aluminium chloride alone is used for precipitation instead of aluminium chloride and calcium chloride, there are obtained aluminium flakes coloured with the aluminium salt of the tetrasulfonic acid of zinc phthalocyanine.

### Examples 13-15

Analogously to Examples 6 to 12, Pigment Red 264 (mono-/di-SO₃H), Pigment Red 264 (di-SO₃H), B1 and PI-VR-0776 (mono-SO₃H) are laked with aluminium and/or calcium. The dyes used in Examples 6 to 15, the amounts of dye and aluminium flakes and the metals used laking are given in the following Table.

| | |
|---|---|
| | B1 |
| 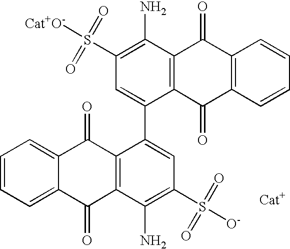 | |

| | | | % alu- | |
|---|---|---|---|---|
| Exam- | | % | minium | |
| ple | Dye | dye | flakes | Laking |
| 6 | Pigment Yellow 191 (di-SO₃H) | 40 | 100 | NH₄⁺/ |
| | | | | Ca²⁺ |
| 7 | Pigment Yellow 191 (di-SO₃H) | 20 | 100 | Al³⁺/Ca²⁺ |
| 8 | Pigment Yellow 191 (di-SO₃H) | 20 | 100 | Al³⁺ |
| 9 | Pigment Red 155 (di-SO₃H/Na) | 30 | 100 | Al³⁺ |
| 10 | Pigment Red 155 (di-SO₃H/Na) | 20 | 100 | Ca²⁺ |
| 11 | copper phthalocyanine (mono- | 50 | 100 | Al³⁺/Ca²⁺ |
| | SO₃H) | | | |
| 12 | zinc phthalocyanine (tetra-SO₃H) | 25 | 100 | Al³⁺ |
| 13 | Pigment Red 264 (mono-/di-SO₃H) | 25 | 100 | Al³⁺ |
| 14 | Pigment Red 264 (di-SO₃H) | 25 | 100 | Al³⁺ |
| 15 | B1 | 25 | 100 | Al³⁺ |
| 16 | C1 | 40 | 100 | Ca²⁺ |

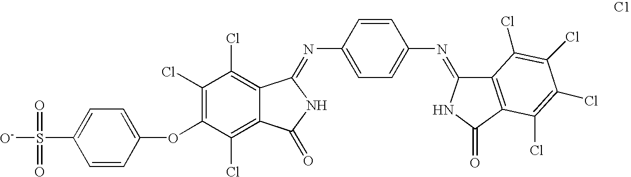

### Example 17

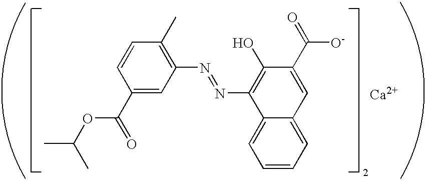

0.30 g of Chromophtal Red 2B is introduced into a 400 ml glass beaker in 100.00 g of deionised water and 10.00 g of methanol and stirred. 0.30 g of sodium hydroxide solution (30%) is introduced and heated at 60° C. for 1 hour. With the heating stopped, further stirring is carried out and the red solution is allowed to cool to room temperature (pH 11.2).

Then, 1.0 g of Aloxal® 3010 (Eckart) is introduced. With vigorous stirring, the pH value is brought to 5.5 within a period of 1 minute, using 3.50 g of 2% hydrochloric acid solution.

Then, 0.13 g of calcium chloride, dissolved in 10 g of water, is slowly added dropwise, the pH value then being 5.4. The dye precipitates out and adheres well to the aluminium flakes. The red suspension is heated to 50° C. for 1 hour, with vigorous stirring. With the heating stopped, the mixture is stirred for a further 2 hours and allowed to cool to room temperature. After filtration and rinsing with 20.00 g of deionised water, the moist filter cake is dried for 16 hours at room temperature and then for 4 hours at 60° C. and 100 hPa, resulting in a red metal effect pigment.

### Examples 18-21

Laking is carried out analogously to Example 17. The dyes used in Examples 17 to 21, the amounts of dye and aluminium flakes and the metals used for laking are given in the following Table.

| | | | % | |
|---|---|---|---|---|
| | | % | aluminium | |
| Example | Dye | dye | flakes | Laking |
| 17 | Pigment Red 220 | 30 | 100 | Ca²⁺ |
| 18 | Pigment Red 221 | 30 | 100 | Ca²⁺ |
| 19 | D1 | 30 | 100 | / |
| 20 | E1 | 30 | 100 | Ca²⁺ |
| 21 | Pigment Yellow 76 | 100 | 100 | / |

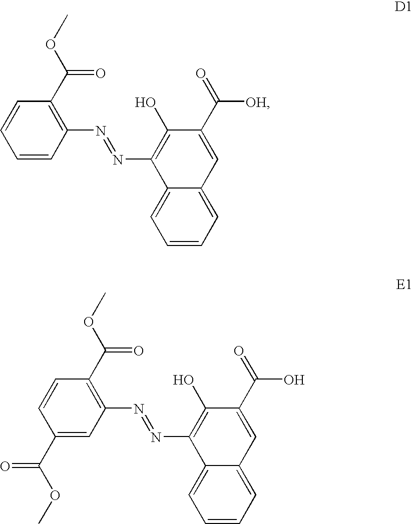

### Example 22

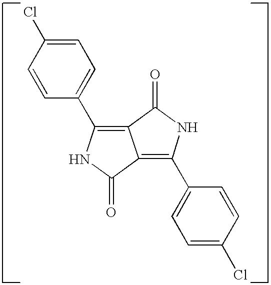

0.60 g of Pigment Red 254 is introduced into a 400 ml glass beaker in 100.00 g of deionised water, 20.00 g of methanol, 2.00 g of sodium silicate solution and 100.00 g of glass beads (diameter: 2 mm) and dispersed for 2 hours. The red suspension is filtered over a glass filter plate and the glass beads are rinsed with 50 g of deionised water. The red suspension is introduced into a 600 ml glass beaker and stirred at room temperature, and 4.00 g of sodium silicate solution are introduced. The pH value is slowly brought to 10.5 by adding 10.0 g of 4% hydrochloric acid solution. Cleaned aluminium flakes (Aloxal® 3010) are then introduced. Whilst stirring vigorously, the pH value is adjusted to 5.8 over a period of 1 minute, using 8.90 g of 4% hydrochloric acid solution.

The mixture is then heated at 50° C. for 1 hour, with stirring. The red pigment is deposited together with sodium silicate in the form of a gel onto the aluminium flakes. The pH value is 6.6 and is slowly brought to 3.1, using 0.40 g of 4% hydrochloric acid solution. Then, 5% sodium stearate solution (0.07 g of sodium stearate dissolved at 50° C. in 10 g of water) is slowly added dropwise, whereupon the pH value is 3.7; the mixture is stirred vigorously for 10 minutes, then stirred for a further 2 hours with the heating stopped, and cooled to room temperature. Filtration is carried out using a suction filter, subsequently washing with 200.0 g of deionised water. The moist filter cake is dried first for 16 hours in air at room temperature and then for 4 hours at 60° C. and 100 hPa in a vacuum drying cabinet, resulting in a red metal effect pigment.

### Examples 23-28

Coating is performed analogously to Example 22. The pigments used in Examples 22 to 28 and the amounts of pigment and aluminium flakes are given in the following Table.

| | | | % | |
|---|---|---|---|---|
| | | % | aluminium | |
| Example | Pigment | pigment | flakes | Coating |
| 22 | Pigment Red 254 | 75 | 100 | 5% sodium |
| | | | | stearate |
| 23 | Pigment Red 179 | 100 | 100 | 5% sodium |
| | (Irgazin ® Red 2273) | | | stearate |
| 24 | Pigment Red 179 | 100 | 100 | 5% sodium |
| | (Irgazin ® Maroon | | | stearate |
| | 3379) | | | |
| 25 | Pigment Red 264 | 100 | 100 | 5% sodium |
| | | | | stearate |
| 26 | Pigment Yellow 109 | 100 | 100 | 5% sodium |
| | | | | stearate |
| 27 | Pigment Yellow 110 | 100 | 100 | 5% sodium |
| | | | | stearate |
| 28 | Pigment Blue 15:3; | 80 | 100 | 5% sodium |
| | copper phthalocyanine | | | stearate |
| | (β) | | | |

1. A method of producing colored carrier particles, which comprises
a) dispersing a pigment in aqueous solution,
b) adding soda waterglass,
c) precipitating SiO₂ and the pigment onto the carrier particles by lowering the pH value.

2. Colored carrier particles obtained by the method according to claim 1.

3. Textiles, surface-coating compositions, printing inks, plastics, glass, ceramic products, cosmetic preparations comprising the colored carrier particles according to claim 2.

4. A method of producing colored carrier particles according to claim 1, wherein carrier particles are stirred into a mixture resulting from steps a and b.

5. A method of producing colored carrier particles according to claim 1, wherein the carrier particles are selected from the group consisting of metallic, metal oxide, non-metallic or non-metal oxide effect pigments, organic pigments and inorganic pigments.

6. A method of producing colored carrier particles according to claim 1, wherein the carrier particles are selected from group consisting of aluminium flakes of pure aluminium or aluminium alloys, copper flakes, copper/tin flakes, copper/zinc flakes, titanium, silver, zinc, tin, stainless steel, effect pigments comprising SiOₓ (0.03≦x≦0.95) and effect pigments comprising SiOₓ (0.95<x≦2.0).

7. A method of producing colored carrier particles according to claim 1, wherein the pigment of step a is selected from 1-aminoanthraquinone, anthraquinone, anthrapyrimidine, azo, azomethine, benzodifuranone, quinacridone, quinacridone quinone, quinophthalone, diketopyrrolopyrrole, dioxazine, flavanthrone, indanthrone, indigo, isoindoline, isoindolinone, isoviolanthrone, perinone, perylene, phthalocyanine, pyranthrone and thioindigo pigments.

8. Colored carrier particles obtained by the method according to claim 5.

9. Colored carrier particles obtained by the method according to claim 6.
